(19) United States
(12) Patent Application Publication
(10) Pub. No.: US 2024/0209333 A1
(43) Pub. Date: Jun. 27, 2024

(54) MULTICOMPONENT XNAZYME-BASED NUCLEIC ACID DETECTION SYSTEM

(71) Applicant: THE REGENTS OF THE UNIVERSITY OF CALIFORNIA, Oakland, CA (US)

(72) Inventors: John Chaput, Irvine, CA (US); Kefan Yang, Irvine, CA (US)

(21) Appl. No.: 18/558,122

(22) PCT Filed: May 2, 2022
(86) PCT No.: PCT/US22/27251

§ 371 Date: Oct. 30, 2023

Related U.S. Application Data
(60) Provisional application No. 63/182,231, filed on Apr. 30, 2021.

(51) Int. Cl. C12N 9/22; C12Q 1/6816

(52) U.S. Cl. CPC C12N 9/22; C12Q 1/6816; C12N 2310/127; C12N 2310/3231

(57) ABSTRACT

XNAzyme compositions featuring nucleotide analogs, wherein the compositions are capable of rapid, inexpensive, sensitive, and accurate nucleic acid detection. The methods, systems, and compositions herein can provide new options for pathogen detection (e.g., virus detection such as but not limited to SARS-CoV-2), disease diagnosis, and genotyping. The XNAzyme compositions of the present invention combine analyte preamplification with X10-23 mediated catalysis to detect particular nucleic acid trigger sequences. The system functions with a detection limit of at least 20 aM (˜10 copies/μL). With an assay time of less than an hour, the present invention provides a faster alternative to quantitative real-time PCR used for viral detection.

### CROSS-REFERENCES TO RELATED APPLICATIONS

This application claims benefit of U.S. Provisional Application No. 63/182,231 filed Apr. 30, 2021, the specification of which is incorporated herein in their entirety by reference.

### REFERENCE TO A SEQUENCE LISTING

Applicant asserts that the information recorded in the form of an Annex C/ST.25 text file submitted under Rule 13ter.1(a), entitled >>>UCI 21.09 PCT Sequencing Listing_ST25<<<, is identical to that forming part of the international application as filed. The content of the sequence listing is incorporated herein by reference in its entirety.

### FIELD OF THE INVENTION

The present invention relates to isothermal amplification methods and systems for rapid and accurate nucleic acid detection. The present invention may be used to detect nucleic acid such as that of pathogens (e.g., viral pathogens, bacterial pathogens, etc.), or for other purposes such as but not limited to genotyping.

### BACKGROUND OF THE INVENTION

Routine, large-scale testing capacity is needed to control the spread of pathogenic diseases such as SARS-CoV-2. Quantitative reverse transcription real-time PCR (qRT-PCR) is the gold standard analytical technique for detecting the SARS-CoV-2 virus in patients. However, it can be associated with slow turnaround times (>24 hours), limited scalability, and the need for specialized equipment, reagents, and trained personnel. Technologies such as reverse transcription-loop mediated isothermal amplification (RT-LAMP) and reverse-transcription-recombinase polymerase amplification (RT-RPA) have been associated with problems with non-specific DNA amplification, leading to high rates of false positive diagnosis.

### BRIEF SUMMARY OF THE INVENTION

The present invention provides a nucleic acid detection platform called “RNA encoded viral nucleic acid analyte reporter” (as used herein, “REVEALR”). The methods, systems, and compositions of the present invention are not limited to RNA detection and also include the detection of DNA, nucleic acid chimeras, or nucleic acids featuring analogs.

The methods, systems, and compositions of the present invention feature a multicomponent (e.g., split) nucleic enzyme (e.g., XNAzyme) derived from DNAzyme 10-23, wherein the XNAzyme comprises nucleic acid analogues. The XNAzyme features a split configuration, enabling its function as a sensor capable of generating an output signal in response to the presence of an input trigger sequence (nucleic acid sequence). Signal amplification via cleavage of a reporter (e.g., nucleic acid reporter) occurs when the XNAzyme is bound to the trigger sequence, allowing for highly specific monitoring of nucleic acid.

Inventors surprisingly discovered that the XNAzyme of the present invention, featuring nucleic acid analogues, allows for the detection of nucleic acid (e.g., RNA, viral RNA, DNA etc.) with attomolar (aM) sensitivity.

The XNAzyme compositions herein (e.g., the sensor) may be engineered to recognize any genetic signal of interest. Non-limiting examples of genetic signals of interest may include but are not limited to viral nucleic acid, bacterial nucleic acid, pathogen nucleic acid, genotypes (e.g., disease genotypes) for humans or other animals, for example for detecting cancers, mutations related to enhancement of infectivity of human or animal pathogens, etc. Thus, the present invention may be used for detection or diagnostic purposes, such as point-of-care diagnostics, and the present invention may be used for genotyping as well. Non-limiting examples of applications include detection or diagnosis of respiratory infections, e.g., SARS-CoV-2 detection, etc., and the genotyping of a particular SARS-CoV-2 strain. The methods and systems of the present invention may help curb the spread of pathogens, such as pathogens associated with pandemics, by providing a rapid and inexpensive point-of-care diagnostic system for routine healthcare monitoring.

Without wishing to limit the present invention to any theory or mechanism, it is believed that the present invention is advantageous because it benefits from greater targetability of the genome sequence as there is no need for a guide sequence as is required for CRISPR. Likewise, because the present invention avoids the need for recombinant protein expression of the Cas protein, it is technically simpler, cheaper, more scalable, and less expensive to implement.

Without wishing to limit the present invention to any theory or mechanism, it is believed that the methods, systems, and compositions herein are advantageous and provide strategies capable of rapid, inexpensive, and accurate nucleic acid detection.

The present invention is also advantageous because its sensitivity allows for detection of very small amounts of nucleic acid.

The present invention provides a multicomponent nucleic acid enzyme composition comprising a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end; and a second nucleic enzyme component comprising a second nucleic acid catalytic core according to SEQ ID NO: 2 (5′-3′ GGCTACGU), SEQ ID NO: 3 (5′-3′ GGCTACGT), or SEQ ID NO: 4 (5′-3′ GGCTAGCT) flanked by a second nucleic acid substrate binding arm at its 5′ end and a second nucleic acid trigger arm at its 3′ end, wherein the residues of SEQ ID NO: 2, SEQ ID NO: 3, or SEQ ID NO: 4 at position 2 and 8 are 2′-fluoroarabino nucleic acid (FANA) residues. Upon assembly of the first nucleic acid catalytic core and second nucleic acid catalytic core, the first nucleic acid catalytic core and second nucleic acid catalytic core form an active enzyme for cleaving nucleic acid. In some embodiments, at least 50% of residues of the first nucleic acid substrate binding arm and at least 50% of residues of the first nucleic acid trigger arm are nucleic acid analogues; and at least 50% of residues of the second nucleic acid substrate binding arm and at 50% of residues of the second nucleic acid trigger arm are nucleic acid analogues.

The present invention provides a multicomponent nucleic acid enzyme composition comprising a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end; and a second nucleic enzyme component comprising a second nucleic acid catalytic core according to SEQ ID NO: 2 (5′-3′ GGCTACGU), SEQ ID NO: 3 (5′-3′ GGCTACGT), or SEQ ID NO: 4 (5′-3′ GGCTAGCT) flanked by a second nucleic acid substrate binding arm at its 5′ end and a second nucleic acid trigger arm at its 3′ end, wherein the residues of SEQ ID NO: 2, SEQ ID NO: 3, or SEQ ID NO: 4 at position 2 and 8 are nucleic acid analogues. Upon assembly of the first nucleic acid catalytic core and second nucleic acid catalytic core, the first nucleic acid catalytic core and second nucleic acid catalytic core form an active enzyme for cleaving nucleic acid. In some embodiments, at least 50% of residues of the first nucleic acid substrate binding arm and at least 50% of residues of the first nucleic acid trigger arm are nucleic acid analogues; and at least 50% of residues of the second nucleic acid substrate binding arm and at 50% of residues of the second nucleic acid trigger arm are nucleic acid analogues.

The present invention provides a multicomponent nucleic acid enzyme composition comprising a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end; and a second nucleic enzyme component comprising a second nucleic acid catalytic core according to SEQ ID NO: 2 (5′-3′ GGCTACGU), SEQ ID NO: 3 (5′-3′ GGCTACGT); wherein residue of SEQ ID NO: 3 at position 8 is a nucleic acid analogue, or SEQ ID NO: 4 (5′-3′ GGCTAGCT) flanked by a second nucleic acid substrate binding arm at its 5′ end and a second nucleic acid trigger arm at its 3′ end, wherein at least 10% of residues of the second nucleic acid trigger arm are nucleic acid analogue. Upon assembly of the first nucleic acid catalytic core and second nucleic acid catalytic core, the first nucleic acid catalytic core and second nucleic acid catalytic core form an active enzyme for cleaving nucleic acid.

In some embodiments, at least 5%, or at least 10%, or at least 15%, or at least 20%, or at least 30% or at least 40% of the nucleic acid trigger arm (e.g., the second nucleic acid trigger arm) comprises nucleic acid analogues (e.g., locked nucleic acids (LNA)). In other embodiments, at least 1 residue, or at least 2 residues, or at least 3 residues, or at least 4 residues, or at least 5 residues of the nucleic acid trigger arm (e.g., the second nucleic acid trigger arm) comprises nucleic acid analogues (e.g., locked nucleic acids (LNA)). In some embodiments, residues of the nucleic acid trigger arm (e.g., the second nucleic acid trigger arm) at positions 5 and/or 6 are nucleic acid analogue (e.g., LNA)). For genotyping, the nucleic acid analogue (e.g., LNA) of the nucleic acid trigger arm (e.g., the second nucleic acid trigger arm) may be positioned such that the nucleic acid analogue (e.g., LNA) aligns with the position of the SNP in the genome being genotyped.

In some embodiments, the nucleic acid analogue is selected from: 2′-fluoroarabino nucleic acid (FANA), locked nucleic acid (LNA), peptide nucleic acid (PNA), hexose nucleic acid (HNA), threose nucleic acid (TNA), cyclohexenyl nucleic acid (CeNA), morpholino nucleic acid (MNA), a 2′ substituted RNA, a sugar modified analog (e.g., sugar modified analog developed for antisense oligonucleotides) and unnatural base pair (UBP). In some embodiments, the 2′ substituted RNA is (OCH3), 2′ amino (NH2), 2′ methoxyethoxy (MOE), 2′-O-methylations (2′-O-Me), 2′ Fluoro, or the like. In some embodiments, the sugar modified analog is Me-ANA, MOE-ANA, 6′-methyl F-HNA, or the like. In some embodiments, the unnatural base pair (UBP) is 2-amino-8-(2-thienyl)purine (s) and pyridine-2-one (y), 7-(2-thienyl)imidazo[4,5-b]pyridine (Ds) and pyrrole-2-carbaldehyde (Pa), Ds and 4-[3-(6-aminohexanamido)-1-propynyl]-2-nitropyrrole (Px), or the like.

In some embodiments, none (e.g., 0%) of residues of the first nucleic acid substrate binding arm and/or none (e.g., 0%) of residues of the first nucleic acid trigger arm are nucleic acid analogues. In some embodiments, none (e.g., 0%) of residues of the second nucleic acid substrate binding arm and/or none (e.g., 0%) of residues of the second nucleic acid trigger arm are nucleic acid analogues. In some embodiments, at least 10% of residues of the first nucleic acid substrate binding arm and/or at least 10% of residues of the first nucleic acid trigger arm are nucleic acid analogues. In some embodiments, at least 10% of residues of the second nucleic acid substrate binding arm and/or at 10% of residues of the second nucleic acid trigger arm are nucleic acid analogues. In some embodiments, at least 25% of residues of the first nucleic acid substrate binding arm and/or at least 25% of residues of the first nucleic acid trigger arm are nucleic acid analogues. In some embodiments, at least 25% of residues of the second nucleic acid substrate binding arm and/or at 25% of residues of the second nucleic acid trigger arm are nucleic acid analogues. In some embodiments, at least 50% of residues of the first nucleic acid substrate binding arm and/or at least 50% of residues of the first nucleic acid trigger arm are nucleic acid analogues. In some embodiments, at least 50% of residues of the second nucleic acid substrate binding arm and/or at 50% of residues of the second nucleic acid trigger arm are nucleic acid analogues. In some embodiments, at least 75% of residues of the first nucleic acid substrate binding arm and/or at least 75% of residues of the first nucleic acid trigger arm are nucleic acid analogues. In some embodiments, at least 75% of residues of the second nucleic acid substrate binding arm and/or at 75% of residues of the second nucleic acid trigger arm are nucleic acid analogues. In some embodiments, at least 80% of residues of the first nucleic acid substrate binding arm and/or at least 80% of residues of the first nucleic acid trigger arm are nucleic acid analogues. In some embodiments, at least 80% of residues of the second nucleic acid substrate binding arm and/or at 80% of residues of the second nucleic acid trigger arm are nucleic acid analogues. In some embodiments, at least 90% of residues of the first nucleic acid substrate binding arm and/or at least 90% of residues of the first nucleic acid trigger arm are nucleic acid analogues. In some embodiments, at least 90% of residues of the second nucleic acid substrate binding arm and/or at 90% of residues of the second nucleic acid trigger arm are nucleic acid analogues. In some embodiments, at least 95% of residues of the first nucleic acid substrate binding arm and/or at least 95% of residues of the first nucleic acid trigger arm are nucleic acid analogues. In some embodiments, at least 95% of residues of the second nucleic acid substrate binding arm and/or at 95% of residues of the second nucleic acid trigger arm are nucleic acid analogues. In some embodiments, all of the residues of the first nucleic acid substrate binding arm and/or all (e.g. 100%) of the residues of the first nucleic acid trigger arm are nucleic acid analogues. In some embodiments, all of the residues of the second nucleic acid substrate binding arm and/or all (e.g., 100%) of the residues of the second nucleic acid trigger arm are nucleic acid analogues.

In some embodiments, at least 5%, at least 10%, or at least 25%, or at least 50%, or at least 75%, or at least 80%, or at least 90%, or at least 100% of the residues of the trigger arms and substrate binding arms are nucleic acid analogs. In some embodiments, all of the residues of the trigger arms and substrate binding arms are nucleic acid analogs. In other embodiments, about 5%, about 10%, or about 25%, or about 50%, or about 75%, or about 80%, or about 90%, or about 100% of the residues of the trigger arms and substrate binding arms are nucleic acid analogs.

In some embodiments, the first nucleic enzyme component comprises a first nucleic acid catalytic core according to SEQ ID NO: 1 (5′-3′ ACAACGA) or SEQ ID NO: 2 (5′-3′ GGCTACGU) or SEQ ID NO: 3 (5′-3′ GGCTACGT) or SEQ ID NO: 4 (GGCTAGCT). In some embodiments, the second nucleic enzyme component comprising a second nucleic acid catalytic core according to SEQ ID NO: 1 (5′-3′ ACAACGA) or SEQ ID NO: 2 (5′-3′ GGCTACGU) or SEQ ID NO: 3 (5′-3′ GGCTACGT) or SEQ ID NO: 4 (GGCTAGCT). In some embodiments, the first nucleic acid catalytic core and/or the second nucleic acid catalytic core may comprise nucleic acid analogues. In some embodiments, the first nucleic acid catalytic core and/or the second nucleic acid catalytic core may comprise at least 2 nucleic acid analogues. In some embodiments, the first nucleic acid catalytic core and/or the second nucleic acid catalytic core may comprise nucleic acid analogues at position 2 and/or position 8.

TABLE 4
Non-limiting examples of Nucleic Acid Catalytic
Core Sequences. Bolded residues indicate
residues that are nucleic acid analogues.

| Sequence | SEQ |
|---|---|
| 5′-3 | ID NO: |
| ACAACGA | 1 |
| GGCTACGU | 2 |
| GGCTACGT | 3 |
| GGCTAGCT | 4 |
| GGCTACGU | 5 |
| GGCTACGT | 6 |
| GGCTAGCT | 7 |
| GGCTACGU | 8 |
| GGCTACGT | 9 |
| GGCTAGCT | 10 |
| GGCTACGU | 11 |
| GGCTACGT | 12 |
| GGCTAGCT | 13 |

TABLE 5
Non-limiting examples of Nucleic Acid Catalytic
Core Sequences. Bolded residues indicate
residues that are 2′-fluoroarabino nucleic
acid (FANA) residues.

| Sequence | SEQ |
|---|---|
| 5′-3 | ID NO: |
| GGCTACGU | 14 |
| GGCTACGT | 15 |
| GGCTAGCT | 16 |
| GGCTACGU | 17 |
| GGCTACGT | 18 |
| GGCTAGCT | 19 |
| GGCTACGU | 20 |
| GGCTACGT | 21 |
| GGCTAGCT | 22 |

In some embodiments, the composition assembles into an active enzyme in the presence of a trigger sequence (e.g., nucleic acid trigger sequence). In some embodiments, the nucleic acid is RNA, DNA, or a combination thereof. In some embodiments, the trigger sequence comprises a nucleic acid having a particular sequence, wherein the trigger arms are complementary to the trigger sequence.

In some embodiments, the composition is a nucleic acid sensor. In some embodiments, the composition is for detecting nucleic acid of a pathogen, e.g., a virus, bacterium, a fungus, a protozoan, a parasite, etc. In some embodiments, the composition is for genotyping.

In some embodiments, the composition is capable of generating an output signal in response to detection of the trigger sequence. In some embodiments, the active enzyme can cleave a nucleic acid reporter. In some embodiments, cleaving the nucleic acid reporter generates a detectable signal. In some embodiments, the active enzyme cleaves the nucleic acid reporter upon detection of the trigger sequence. In some embodiments, the composition can be engineered to detect a specific trigger sequence. In some embodiments, the composition can be engineered to detect a specific nucleic acid reporter.

In some embodiments, the composition is capable of analyte detection of <20 aM. In some embodiments, the composition is capable of analyte detection of <50 aM. In some embodiments, the composition can detect attomolar levels of a trigger sequence in less than 1 hour.

In some embodiments, the first substrate binding arm and/or the second substrate binding arm are at least 5 nucleotides in length. In some embodiments, the first substrate binding arm and/or the second substrate binding arm are at least 6 nucleotides in length. In some embodiments, the first substrate binding arm and/or the second substrate binding arm are at least 7 nucleotides in length. In some embodiments, the first substrate binding arm and/or the second substrate binding arm are at least 8 nucleotides in length. In some embodiments, the first substrate binding arm and/or the second substrate binding arm are at least 9 nucleotides in length. In some embodiments, the first substrate binding arm and/or the second substrate binding arm are at least 10 nucleotides in length. In some embodiments, the first substrate binding arm and/or the second substrate binding arm are at least 11 nucleotides in length. In some embodiments, the first substrate binding arm and/or the second substrate binding arm are at least 12 nucleotides in length. In some embodiments, the first substrate binding arm and/or the second substrate binding arm are at least 15 nucleotides in length. In some embodiments, the first substrate binding arm and/or the second substrate binding arm are about 5 to 10 nucleotides in length, or about 5 to 15 nucleotides in length. In other embodiments, the first substrate binding arm and/or the second substrate binding arm are about 5 nucleotides, or about 6 nucleotides, or about 7 nucleotides, to about 8 nucleotides, or about 9 nucleotides, or about 10 nucleotides, or about 11 nucleotides, or about 12 nucleotides, or about 13 nucleotides, or about 14 nucleotides, or about 15 nucleotides in length. In further embodiments, length of the first substrate binding arm and/or the second substrate binding arm varies depending on the substrate sequence and the incubation temperature.

In some embodiments, the first trigger arm and/or second trigger arm are at least 5 nucleotides in length. In some embodiments, the first trigger arm and/or second trigger arm are at least 6 nucleotides in length. In some embodiments, the first trigger arm and/or second trigger arm are at least 8 nucleotides in length. In some embodiments, the first trigger arm and/or second trigger arm are at least 10 nucleotides in length. In some embodiments, the first trigger arm and/or second trigger arm are at least 15 nucleotides in length. In some embodiments, the first trigger arm and/or second trigger arm are at least 20 nucleotides in length. In some embodiments, the first trigger arm and/or second trigger arm are about 5 nucleotides, or about 6 nucleotides, or about 7 nucleotides, or about 8 nucleotides, or about 10 nucleotides, or about 15 nucleotides, or about 20 nucleotides in length.

The present invention also provides a reporter composition, e.g., a nucleic acid reporter composition. In some embodiments, the composition comprises an oligonucleotide having a cleavage site, a label disposed on one side of the cleavage site and a masking molecule disposed on one side of the cleavage site opposite the label, wherein the masking molecule prevents the label from being detectable when the nucleic acid reporter is not cleaved, and the label is detectable when the nucleic acid reporter is cleaved at its cleavage site. In some embodiments, the nucleic acid reporter comprises RNA. In some embodiments, the nucleic acid reporter comprises DNA. In some embodiments, the nucleic acid reporter comprises a chimera of RNA and DNA. In some embodiments, the nucleic acid reporter comprises RNA, DNA, a chimera of RNA and DNA, a nucleic acid analog, or a combination thereof. In some embodiments, the nucleic acid analogue is selected from: 2′-fluoroarabino nucleic acid (FANA), locked nucleic acid (LNA), peptide nucleic acid (PNA), hexose nucleic acid (HNA), threose nucleic acid (TNA), cyclohexenyl nucleic acid (CeNA), morpholino nucleic acid (MNA), a 2′ substituted RNA, and a sugar modified analog (e.g., sugar modified analog developed for antisense oligonucleotides). In some embodiments, the 2′ substituted RNA is (OCH3), 2′ amino (NH2), 2′ methoxyethoxy (MOE), or the like. In some embodiments, the sugar modified analog is Me-ANA, MOE-ANA, 6′-methyl F-HNA, or the like. In some embodiments, the label is a fluorescent label. In some embodiments, the label is a colorimetric label. In some embodiments, the masking molecule is a quencher. In some embodiments, the masking molecule is biotin.

The present invention also provides a kit comprising a multicomponent enzyme composition according to the present invention and a nucleic acid reporter as described herein. In some embodiments, the enzyme composition becomes active upon detection of a trigger sequence, the nucleic acid reporter is cleaved when the enzyme composition becomes active, and the label of the nucleic acid reporter becomes detectable when the nucleic acid reporter is cleaved.

The present invention also provides a system comprising a multicomponent enzyme composition according to the present invention and a nucleic acid reporter as described herein. In some embodiments, the enzyme composition becomes active upon detection of a trigger sequence, the nucleic acid reporter is cleaved when the enzyme composition becomes active, and the label of the nucleic acid reporter becomes detectable when the nucleic acid reporter is cleaved. In some embodiments, the system is a sequence-specific detection system wherein the multicomponent enzyme composition converts an input signal into an observable output signal. In some embodiments, the input signal is the trigger sequence. In some embodiments, the output signal can be read by fluorescence technology. In some embodiments, the output signal can be read by colorimetric technology. In some embodiments, the output signal can be read by lateral flow systems.

The present invention also provides a point-of-care detection system. In some embodiments, the system comprises a multicomponent enzyme composition according to the present invention; a reporter composition, and a platform for applying amplifying a target nucleic acid sequence to generate a plurality of trigger sequences. In some embodiments, the platform is further for applying the enzyme composition and nucleic acid reporter to the plurality of trigger sequences for detection of the target nucleic acid. In some embodiments, the target nucleic acid is a nucleic acid of a pathogen, e.g., a virus, a bacterium, a fungus, a paradise, a protozoan, etc. In some embodiments, the target nucleic acid is for genotyping.

The present invention also provides a method of detecting nucleic acid. In some embodiments, the method comprises amplification of a target nucleic acid; and making detectable the amplified target nucleic acid. In some embodiments, amplification of the nucleic acid comprises RPA or RT-RPA followed by T7 transcription of amplified nucleic acid to generate trigger sequences. In some embodiments, the trigger sequences are single-stranded RNA triggers. In some embodiments, detection of the amplified nucleic acid comprises introducing a multicomponent nucleic acid enzyme composition according to the present invention and a reporter composition. In some embodiments, the enzyme composition assembles on the RNA triggers. In some embodiments, the assembled enzyme cleaves the nucleic acid reporter to make the trigger sequences detectable, which is indicative of the target nucleic acid. In some embodiments, the target nucleic acid is a nucleic acid of a pathogen, e.g., a virus, a bacterium, a fungus, a paradise, a protozoan, etc. In some embodiments, the target nucleic acid is for genotyping.

The present invention also provides a method of detecting nucleic acid in a sample. In some embodiments, the method comprises subjecting the sample to amplification for amplifying a target nucleic acid via production of trigger sequences; and introducing to the sample a multicomponent enzyme according to the present invention and a nucleic acid reporter; wherein the enzyme binds to the trigger sequences and becomes active, wherein the active enzyme cleaves the nucleic acid reporter to generate a detectable signal. In some embodiments, the amplification is RPA or RT-RPA and transcription with T7 RNA polymerase. In some embodiments, the target nucleic acid is a nucleic acid of a pathogen (e.g., a virus, a bacterium, a fungus, a paradise, a protozoan, etc.). In some embodiments, the target nucleic acid is for genotyping. In some embodiments, the method is for detecting a mutation in a sequence. In some embodiments, the mutation is associated with a disease or condition (e.g., cancer, etc.). In some embodiments, the detectable signal quantifies the target nucleic acid in the sample.

The present invention also provides a method of detecting SARS-CoV-2. In some embodiments, the method comprises subjecting the sample to amplification for amplifying a target RNA sequence of SARS-CoV-2; and introducing to the sample a multicomponent enzyme composition according to the present invention and a reporter, wherein the enzyme binds to the amplified target RNA thereby activating the enzyme, whereupon the enzyme cleaves the reporter to generate a detectable signal. In some embodiments, the detectable signal is indicative of detecting SARS-CoV-2 in the sample. In some embodiments, the reporter is a fluorescent reporter. In some embodiments, the method further comprises genotyping the SARS-CoV-2 strain.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1A shows a schematic view of a multicomponent nucleic acid sensor comprising two catalytic cores (left and right, labelled Mz-A and Mz-B). The two catalytic cores self-assemble in the presence of a nucleic acid trigger (e.g., RNA, etc.) to produce a functionally active catalyst with a separate enzyme active site that is capable of site-specific leverage of an RBA reporter. Color code: RNA (red), DNA (black), FANA (orange/dots), input trigger (green).

FIG. 1B shows the chemical structures of the nucleic acid molecules used in the nucleic acid sensor described herein.

FIG. 1C shows a specific example of a nucleic acid sequence for the multicomponent sensor designed to detect the S1 protein coding region of SARS-CoV-2. Red arrow indicates G-U dinucleotide cleavage site. Color code: DNA (black), RNA (red), and FANA (orange). Sequences: Mz-A CACCAGCUGUCCAACCUGAAacaacgaGGUUAG (SEQ ID NO: 186); Mz-B UCAUGAggctacguGAAGAAUCACCAGGAGUCAA (SEQ ID NO: 187); Trigger UUGACUCCUGGUGAUUCUUC UUCAGGUUGGACAGCUGGUG (SEQ ID NO: 188); Reporter CUAACCGUCAUGA (189). Notes: Black stands for DNA nucleotides; Underline represents the substrate binding arm; Bold represents a nucleic acid analogue (e.g, FANA analogues).

FIG. 2A shows an overview of the nucleic acid sensor. Two catalytic cores (left and right) self-assemble in the presence of a viral RNA analyte (trigger) to produce a functionally active catalyst with a separate enzyme active site that is capable of site-specific cleavage of an RNA reporter. RNA (red), DNA (black), FANA (orange/dots), input trigger (green). Arrow indicates G-U dinucleotide cleavage site.

FIG. 2B shows steady-state kinetic analysis of RNA cleavage by the multicomponent 10-23, X-10-23, and X10-23 Pro enzymes with representative denaturing polyacrylamide gels showing the formation of a cleaved RNA product over a time span of 0 to 5 min.

FIG. 2C shows the limit of detection for the 10-23, X10-23, and X10-23 Pro multicomponent catalysts measured by fluorescence detection across a range of analyte concentrations. n=3; error bars represent the standard error of the mean, SEM. Two tailed student's t test; *P<0.05, **P<0.01, ***P<0.001, ****P<0.0001. All reactions were performed in a buffer containing 50 mM MgCl2 at pH 8.5 (24° C.). Abbreviations: au, arbitrary units; S, full-length 5′ labeled RNA substrate; P, 5′ cleavage product.

FIG. 3A shows an overview of the detection assay. The SARS-CoV-2 region of interest is isothermally amplified by RT-RPA and then transcribed with T7 RNA polymerase to produce multiple copies of the RNA trigger. X10-23 Pro assembly on the RNA trigger leads to the cleavage of a quenched fluorescent reporter for fluorescence detection or a biotin-labelled fluorescent RNA for lateral flow detection. Cleavage site (arrow).

FIG. 3B shows the kinetics of fluorescent signal generation over 90 min. across a range of RNA dilutions. au, arbitrary units. Measurements are mean±standard error (S.E.M), n=3. Two tailed student's t test; *P<0.05, **P<0.01, ***P<0.001, ****P<0.0001.

FIG. 3C shows the limit of detection for fluorescence-based quantification at 30 min at designated RNA concentrations. Measurements are mean±standard error (S.E.M), n=3. Two tailed student's t test; *P<0.05, **P<0.01, ***P<0.001, ****P<0.0001.

FIG. 3D shows the limit of detection using lateral flow readout containing control and test bands. au, arbitrary units; NTC, no template control.

FIG. 4A shows the kinetics of fluorescence signal generation for a SARS-CoV-2 specific REVEALR assay performed on a range of pseudovirus samples.

FIG. 4B shows the fluorescence signal generated after 60 min for a SARS-CoV-2 specific REVEALR assay. Two tailed student's t test; *P<0.05, **P<0.01, ***P<0.001, ****P<0.0001. Threshold is defined as twice the signal-to-noise (S/N) of the NTC fluorescence intensity. Samples were poised at 50 fM concentration.

FIG. 4C shows a blinded pseudovirus assay. REVEALR was used to identify SARS-CoV-2 samples in a randomized study. au, arbitrary units. NTC, no template control containing nuclease-free water.

FIGS. 5A and 5B shows a REVEALR-based detection of SARS-CoV-2 variants of concern. FIG. 5A shows the progression of COVID-19 cases in the United States from January 2020 to January 2022. Dominant variants of concern (VOC) observed at the collection date are labeled. Data points are based on a 7-day average. FIG. 5B shows a schematic view of competitive REVEALR genotyping. The SARS-CoV-2 region of interest is isothermally amplified by RT-RPA and T7 RNA polymerase to produce multiple copies of the RNA analyte. Competitive XNAzyme assembly on the viral RNA produces a fluorescent signal specific to the sample genotype via site-specific cleavage of a quenched fluorescent reporter. 2-D analysis shows WT or VOC detection based on the measured fluorescence observed for the FAM and HEX signal, respectively. Abbreviations: WT (wild-type); VOC (variant of concern); X, Y (SNP position); and au (arbitrary units). Arrow indicates substrate cleavage site. Colors: RNA (red); DNA (black); green (WT, Fam signal); and blue (VOC, Hex signal).

FIGS. 6A and 6B shows sensor optimization. FIG. 6A shows an overview of multicomponent nucleic acid sensor. Two catalytic cores (Mz-A and Mz-B) self-assemble in the presence of a viral RNA analyte (not shown) to produce a functionally active catalyst with a separate active site that is capable of site-specific cleavage of a quenched fluorescent RNA reporter. Sensor 1 is DNA, sensor 2 contains a single LNA residue at the SNP position (*), and sensor 3 contains LNA residues at the SNP position (*) and the 5′- and 3′-terminal positions of the substrate binding arms. Red arrow indicates the cleavage site and underlined nucleotides denote LNA residues. FIG. 6B shows sensor optimization. Change in fluorescence observed for sensors 1-3 against decreasing concentration of a matched and mismatched DNA analyte corresponding to the A570D mutation observed in the S1 protein of the SARS-CoV-2 genome. Error bars represent the standard error of the mean (SEM) with n=3. Abbreviations: au, arbitrary units; S, quenched 5′-fluorescent substrate; P, 5′-fluorescent product. Black dot denotes the SNP position.

FIGS. 7A, 7B, and 7C shows the nucleic acid detection assay. FIG. 7A shows fluorescence signals generated by the wild-type and alpha sensors initialized by a segment of the wild-type analyte under non-competitive (left) and competitive (right) reaction conditions. FIG. 7B shows fluorescence signals generated by the wild-type and alpha sensors initialized by a segment of the alpha variant under non-competitive (left) and competitive (right) reaction conditions. Error bars represent the standard error of the mean (SEM) with n=3. FIG. 7C shows a 2-D analysis showing wild-type and alpha variant detection under competitive reaction conditions. Each data point is the average of 3 replicates. NTC, no template control.

FIGS. 8A and 8B shows sensitivity of REVEALR genotyping under competitive conditions. FIG. 8A shows fluorescent signal generation for 15 nM of the A570D, K417N/K417T, L452R, and T547K DNA analyte after incubation for 30 min at 37° C. Measurements are mean standard error (S.E.M), n=3. Two tailed student's t test; *P<0.05, **P<0.01, ***P<0.001, ****P<0.0001. FIG. 8B shows data are presented in 2-D plots showing wild-type and VOC detection across a range of concentrations. Assays were performed by separately delivering either the wild-type or VOC analyte to the reaction mixture. Each data point is the average of 3 replicates.

FIG. 9 shows surveillance testing of SARS-CoV-2 in the United States and Orange County, California. Surveillance of SARS-CoV-2 from GISAID shows the chronological frequency of variants of concern observed in the United States between January 2021 and January 2022 (top plot) using a subsampled dataset with time points documented at 7 day increments. Pie charts depict the distribution of sequence-verified SARS-CoV-2 variants observed in patients treated at UCI Medical Center in Orange, California in early, mid, and late 2021. All 31 clinical samples were positively genotyped by competitive REVEALR. Each data point is the average of 3 replicates. Clinical samples 32-34 are negative controls obtained from healthy patients. Colors: blue, positive for wild-type; green, positive for VOC; yellow, clinical samples that are negative for COVID-19. Abbreviations: W, wild-type; A, alpha; E, epsilon; gamma; delta; and O, omicron; and n/d or -, not detected.

FIG. 10 shows the genotype of SARS-CoV-2 variants. Each grouping contains the codon for an amino acid of interest. The single orange colored letter of the codon represents the SNP mutation evaluated. Red labels to the left and right of the table denote the WHO variant name and Pango lineage, respectively. Mutations given in bold font at the top of the table indicate sensors that were chosen for clinical validation, while regular font indicates the back-up sensors.

FIG. 11 shows a schematic overview of multicomponent enzyme screening. All experiments are performed with 15 nM of the corresponding analyte and incubated at 37° C. for 30 min. WT analyte differs from MT analyte by a single nucleotide in the sequence. WT sensor differs from MT sensor by a single nucleotide in the analyte binding arm of Mz-B. n=3. WT, wild-type. MT, mutant.

FIG. 12 shows back-up sensors identified for SNP discrimination. Fluorescent signal generation under 15 nM of corresponding IVT RNA fragment and incubated at 37° C. for 30 min. Measurements are mean+standard error (S.E.M), n=3. Two tailed student's t-test; *P<0.05, **P<0.01, ***P<0.001, ****P<0.0001. WT, wild-type. MT, mutant.

FIG. 13 shows multicomponent sensors that did not qualify for SNP detection. Fluorescent signal generation under 15 nM of corresponding IVT RNA fragment and incubated at 37° C. for 30 min. Measurements are mean+standard error (S.E.M), n=3. Two tailed student's t-test; *P<0.05, **P<0.01, ***P<0.001, ****P<0.0001. ns, not significant. WT, wild-type. MT, mutant.

FIG. 14 shows eighteen single-nucleotide mutations that were evaluated across all regions of the SARS-CoV-2 genome to establish a panel of multicomponent DNAzymes that could identify each of the major VOCs observed in the population over the last 24 month.

### DETAILED DESCRIPTION OF THE INVENTION

The present invention provides a nucleic acid detection platform called RNA encoded viral nucleic acid analyte reporter, e.g., a multicomponent XNAzyme, for rapidly detecting nucleic acid such as but not limited to RNA (e.g., viral RNA), e.g., DNA, chimeras, etc. The multicomponent XNAzyme may be referred to herein as “REVEALR.” As previously discussed, the present invention is not limited to RNA detection nor viral RNA detection and also includes the detection of DNA. Without wishing to limit the present invention to any theory or mechanism, it is believed that the methods and systems herein are advantageous as they allow for nucleic acid detection with attomolar (aM) sensitivity. In certain embodiments, detection may be achieved in less than an hour.

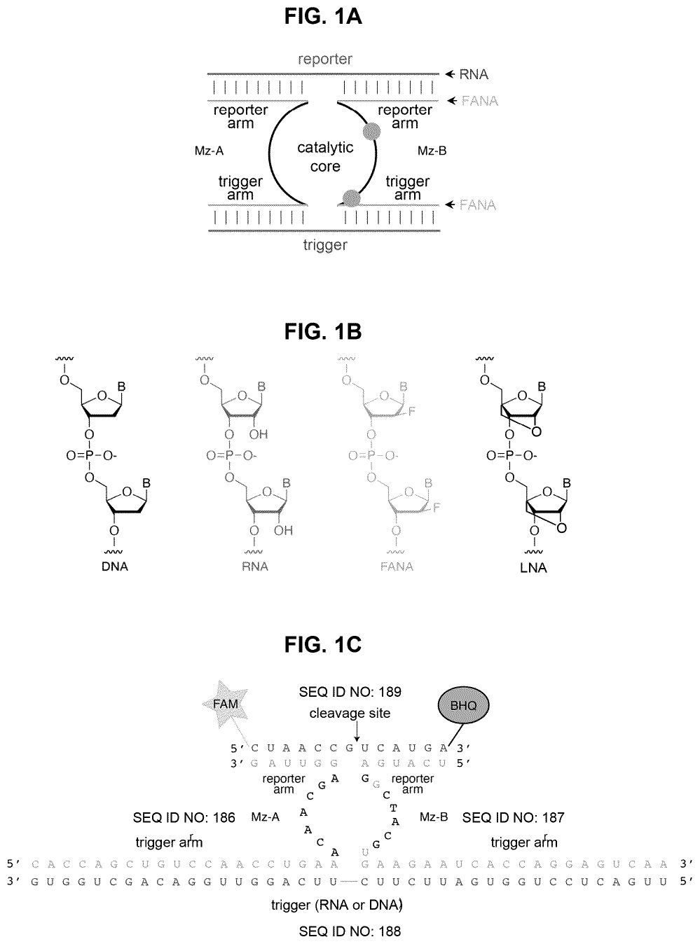

FIG. 1A, FIG. 1B, and FIG. 1C shows examples of the multicomponent XNAzyme of the present invention. The multicomponent XNAzyme is derived from XNAzyme 10-23 and comprises a first catalytic core (e.g., see Mz-A in FIG. 1A) and a second catalytic core (e.g., see Mz-B in FIG. 1A). When assembled, e.g., when bound to the trigger sequence (produced by amplification of a target nucleic acid sequence) the XNAzyme self-assembles into an active form. When the XNAzyme is active, it can cleave a reporter to generate a detectable signal (signal amplification). Thus, the present invention provides a sensor capable of generating an output signal in response to the presence of an input target sequence, e.g., a nucleic acid sequence of interest (trigger sequence). This allows for highly specific detecting and monitoring of the nucleic acid of interest, e.g., single-stranded viral RNA.

The multicomponent XNAzyme comprises nucleic acid substitutions, wherein DNA molecules are replaced with non-natural nucleic acid analogs. For example, positions 2 and 8 of the catalytic core are modified by a nucleic acid analog. Non-limiting examples of nucleic acid analogs include FANA, TNA, LNA, PNA, and HNA.

The multicomponent nucleic acid enzyme can be used to detect and/or quantify nucleic acid. Non-limiting examples of nucleic acids include RNA and DNA, chimeras, or analogs thereof. In certain embodiments, the nucleic acid is that of a virus, bacterium, or other pathogen. The compositions here may also be used to detect particular genotypes.

The enzyme can be engineered to detect a specific target sequence. Likewise, the enzyme can be engineered to detect a specific reporter. In some embodiments, the reporter comprises a fluorescent label. In some embodiments, the reporter comprises a nanoparticle. In some embodiments, the reporter comprises biotin. In some embodiments, the reporter comprises a colorimetric label.

The present invention also provides systems comprising the multicomponent enzyme as disclosed herein and a quenched reporter. The systems may be featured as a kit.

The system provides a sequence-specific detection system wherein the multicomponent enzyme converts an input signal (e.g., a target RNA such as but not limited to viral RNA) into an observable output signal. In certain embodiments, the output signal can be read by fluorescence technology. In certain embodiments, the output signal can be read by colorimetric technology. In certain embodiments, the output signal can be read by lateral flow systems.

The system may be designed as a point-of-care detection system. For example, the system may comprise a multicomponent enzyme according to the present invention; a reporter, and a platform or other tools or reagents for amplification of the target nucleic acid sequence and/or for applying the enzyme composition and reporter to a sample for detection of the target nucleic acid.

As previously discussed, the system may be capable of rapid analyte detection of <20 aM. In certain embodiments, the system can detect the target sequence in less than an hour.

The present invention also provides methods for detecting nucleic acid. In certain embodiments, the method comprises amplification of a target nucleic acid; and making the amplified target nucleic acid detectable. The step of amplifying the nucleic acid may comprise RT-RPA and transcription of amplified RNA to generate single-stranded RNA triggers. The step of making the amplified RNA triggers detectable may comprise introducing the multicomponent nucleic acid enzyme according to the present invention and a reporter. The enzyme can assemble on the RNA triggers and subsequently cleave the reporter to make the amplified RNA detectable (and thus the target nucleic acid).

The present invention also provides methods for detecting SARS-CoV-2. In some embodiments, the method comprises subjecting the sample to amplification for amplifying a target RNA sequence of SARS-CoV-2; and introducing to the sample a multicomponent enzyme according to the present invention and a quenched reporter. The enzyme binds to the amplified target RNA, which activates the enzyme. When active, the enzyme cleaves the quenched reporter to generate a detectable signal, which is indicative of SARS-CoV-2 in the sample.

The methods and systems (e.g., the sensor) may be engineered to recognize any genetic signal (e.g., viral, bacterial, etc.) as well as genotyping of human diseases (e.g., cancers, mutations related to enhancement of infectivity of human or animal pathogens, etc.).

### Example 1: REVEALR

The following is a non-limiting example of the present invention. It is to be understood that said example is not intended to limit the present invention in any way. Equivalents or substitutes are within the scope of the present invention.

The present invention describes isothermal amplification strategies capable of rapid, inexpensive, and accurate viral detection, which may offer an alternative to qRT-PCR as a public health tool for routine virus detection, e.g, SARS-CoV-2 detection.

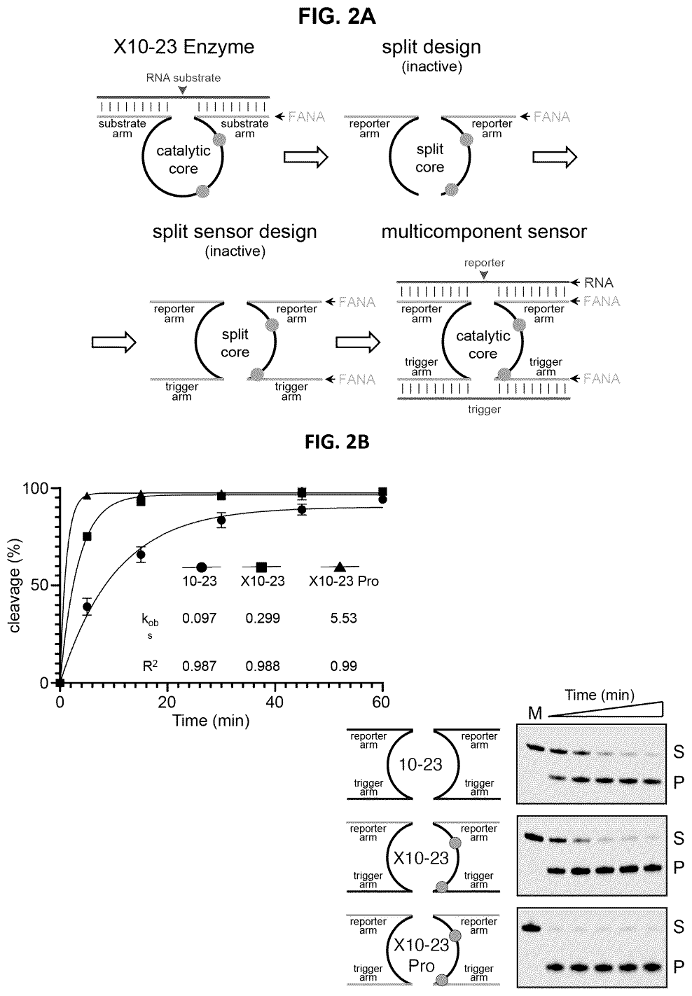

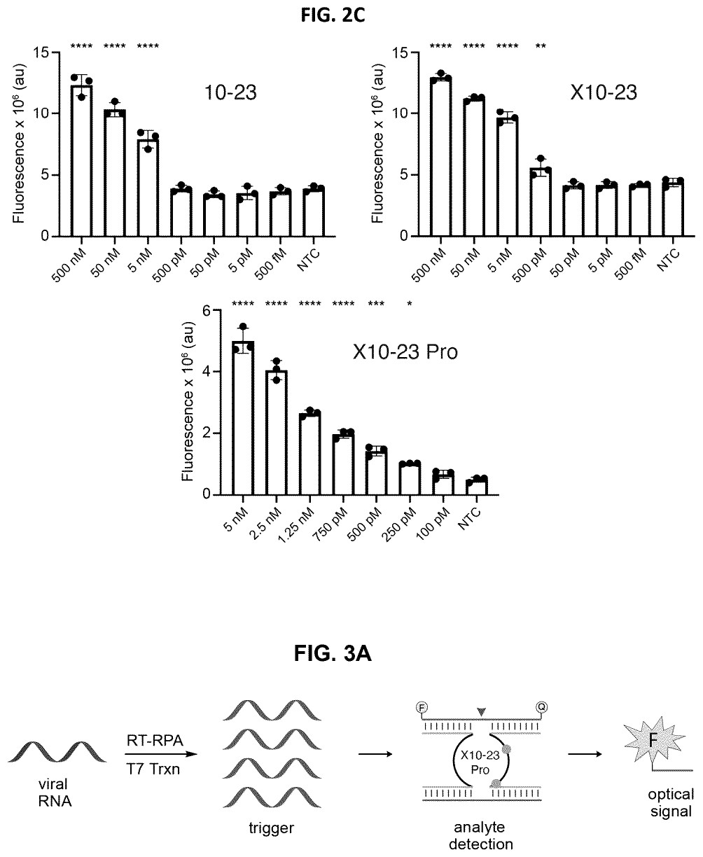

The methods and systems herein may feature the use of XNAzyme 10-23 (or an appropriate alternative). Conversion of X10-23 into a split XNAzyme enables the production of a multicomponent optical sensor (see FIG. 2A) capable of generating an output signal in response to the presence of an input target sequence referred to here as the trigger. Signal amplification via cleavage of a quenched RNA reporter occurs as long as the complex is bound to the target sequence, allowing for highly specific monitoring of single-stranded viral RNA.

To evaluate the multicomponent design, the rate of RNA substrate cleavage for the classic 10-23 DNA design was compared to X10-23 enzyme in the multicomponent format in which the catalytic core of both nucleic acid enzymes was divided into two separate parts that self-assemble into an active sensor in the presence of the viral RNA trigger (see FIG. 2A). Kinetic analysis of both sensors using an RNA reporter indicate that the DNA and XNA catalysts cleave the RNA substrate with pseudo first-order rate constants (k_{obs}) of ˜0.1±0.01 min⁻¹ and 0.3±0.01 min⁻¹ (see FIG. 2B), respectively, when assayed in a buffer containing 50 mM MgCl₂ at pH 8.5 (24° C.). Strikingly, the rate of RNA cleavage by X10-23 increases ˜25-fold when the DNA trigger arms of the X10-23 scaffold are replaced with the unnatural nucleic acid analog 2′-fluoroarabinonucleic acid (FANA). The new multicomponent enzyme, termed X10-23 Pro, functions with an average k_{obs} of 5.5±0.89 min⁻¹ and exhibits a limit of detection (LoD) of 250 pM for in vitro transcribed (IVT) SARS-CoV-2 pseudovirus when evaluated in triplicate (see FIG. 2C).

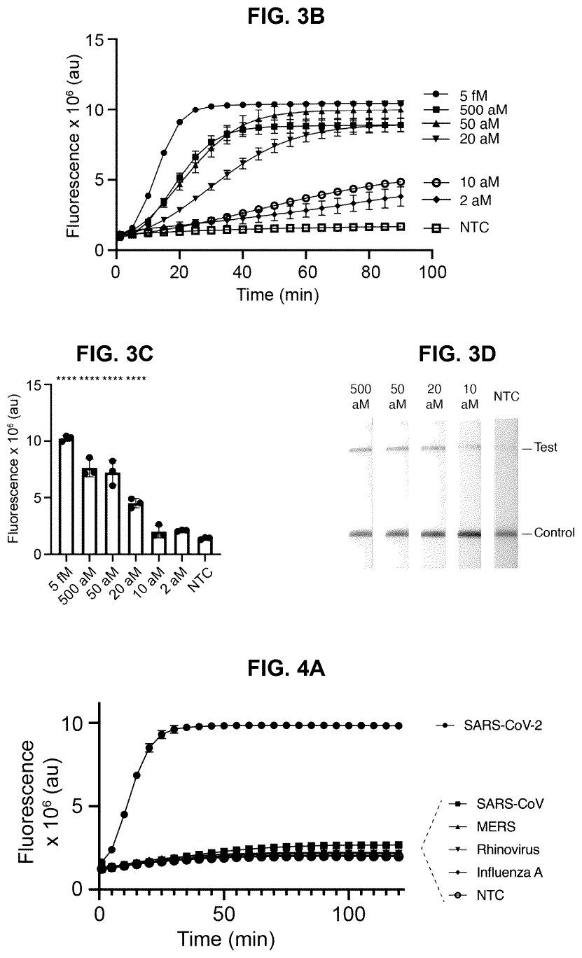

To achieve attomolar (aM) level sensitivity for SARS-CoV-2 detection in human samples, the multicomponent X10-23 Pro system was combined with a preamplification step similar to the one used for CRISPR-based SARS-CoV-2 detection. Accordingly, IVT SARS-CoV-2 RNA pseudovirus was isothermally amplified by RT-RPA and forward transcribed by T7 RNA polymerase to generate the single-stranded RNA trigger required for X10-23 Pro detection. The combined process of viral preamplification with specific nucleic acid detection by X10-23 Pro-mediated hydrolysis of a quenched RNA reporter is referred to as REVEALR (see FIG. 3A).

REVEALR was used to compare the kinetics of fluorescence signal generation for the multicomponent enzymes of 10-23, X10-23, and X10-23 Pro. The assays were performed in a buffer containing a dilution series of quantified SARS-CoV-2 RNA pseudovirus targeting a portion of the viral genome that encodes a region of the spike (S) protein. Kinetic measurements performed in triplicate indicated that X10-23 Pro had the greatest potential for establishing a highly sensitive viral RNA detection assay with an initial LoD of 50 aM. Subsequent optimization of the reaction conditions by adjusting such factors as the magnesium acetate concentration and reverse transcriptase enzyme in the RT-RPA reaction reduced the analytical LoD to 2 aM after 90 min of fluorescence detection (see FIG. 3B).

Using the optimized reaction conditions, the analytic LoD was determined in a 30 minute fluorescence-based assay to be at least 20 aM, which corresponds to ˜10 copies/μL of pseudoviral RNA (see FIG. 3C). This value is below the average viral load observed in COVID-19 patients, which ranges from 80 to 752 copies per μL. The LoD was independently validated using a paper-based lateral flow readout modality (see FIG. 3D), which confirmed an analytical LoD of at least 20 aM. The results from both LoD assays are quantitatively similar to the CRISPR-based systems of SHERLOCK and DETECTR, demonstrating that nucleic acid enzymes can compete with protein enzymes.

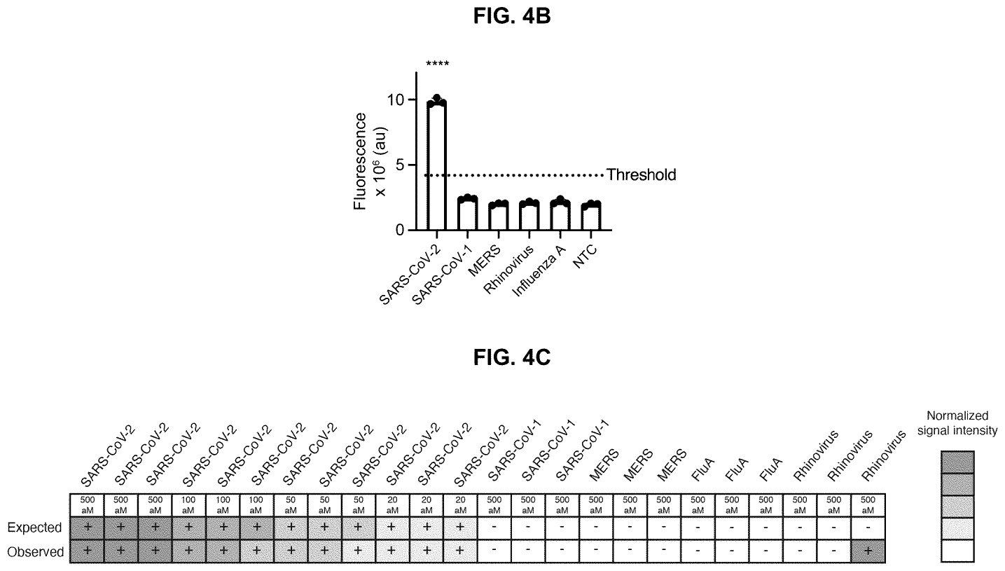

The specificity of REVEALR for SARS-CoV-2 versus other viruses that are known causes of respiratory infections was investigated. IVT RNA pseudovirus was constructed for SARS-CoV-1, MERS, rhinovirus, and influenza A. S-gene specific SARS-CoV-2 REVEALR assays performed on all five viral RNA samples demonstrate that the SARS-CoV-2 assay is rapid (<1 h) and highly specific for SARS-CoV-2 (see FIG. 4). No cross-reactivity was observed for the other four viral samples, including SARS-CoV-1, which is 80% identical to SARS-CoV-2. Real-time and single end point detection assays show that the off-target viral samples are indistinguishable from the no template control (NTC), containing nuclease-free water, all of which have a signal-to-noise (S/N) ratio of less than 2.

Lastly, the REVEALR-based detection system established for SARS-CoV-2 was evaluated in a blinded study of 24-IVT RNA pseudovirus samples. Twelve of the samples contained the SARS-CoV-2 virus poised at concentrations of 20, 50, 100 and 500 aM, while the remaining samples contain SARS-CoV-1, MERS, rhinovirus, or influenza A, each poised at a concentration of 500 aM. The samples were prepared and organized by a team member not affiliated with the study and REVEALR was used to identify the 12 SARS-CoV-2 samples after a 30 minute reaction. Fluorescence analysis shows that REVEALR was able to identify all 12 of the positive samples and 11 of the negative samples, indicating that the assay functions with 100% positive predictive agreement and 92% negative predictive agreement.

Thus, the methods and systems of the present invention (e.g., REVEALR) provide a new strategy to improve the speed, sensitivity, and specificity of pathogen detection, e.g., SARS-CoV-2 detection or other pathogens or nucleic acid (RNA) of interest. Sequence-specific target recognition is achieved using a chemically synthesized multicomponent nucleic acid enzyme that is capable of highly sensitive analyte detection (<20 aM) using an optical or visual readout system that relies on efficient cleavage of an RNA reporter. The present invention provides a programmable nucleic acid platform and a nucleic acid enzyme that can compete with a protein enzyme, making REVEALR an attractive system for pathogen detection.

Materials: DNA and RNA oligonucleotides were purchased from Integrated DNA Technologies (Coralville, IA). DNA and FANA phosphoramidites were purchased from Glen Research Corporation (Sterling, VA). TwistAmp® Basic Kit and TwistAmp® Liquid Basic Kit were purchased from TwistDx (Maidenhead, UK). Solutions of dNTPs (100 mM) and SuperScript IV Reverse transcriptase were purchased from ThermoFisher (Waltham, MA). T7 RNA polymerase and buffer was purchased from Lucigen Corporation (Middleton, WI). HiScribe T7 High Yield RNA Synthesis Kit, RNase H, and M-MuLV Reverse transcriptase were purchased from New England Biolabs (Ipswich, MA). HybriDetect lateral flow strips were purchased from Milenia Biotec (Giessen, DE).

Pseudoviral RNA preparation: DNA versions of the SARS-CoV-2 (S region), SARS-CoV-1, MERS, Rhinovirus, and Influenza A gene fragments were obtained from IDT. All genes were PCR amplified with forward primers containing the T7 promoter. Pseudoviral RNA was then prepared by in vitro transcription using HiScribe T7 High Yield RNA Synthesis Kit. The reaction mixtures contained 10 mM of each NTP, 1× reaction buffer, 3 μL PCR product, 2 μL T7 RNA polymerase mixture and 5 μL of nuclease free water, which were incubated at 37° C. overnight. The crude RNA was purified by 15% denaturing urea PAGE and electroeluted under 180 V for 3 hours. Purified RNA stocks were quantified by NanoDrop and diluted in nuclease-free water to desired concentrations.

Oligonucleotide synthesis: Synthetic oligonucleotides containing FANA residues were synthesized in-house using an automated solid-phase DNA synthesizer (Applied Biosystems 3400) on Glen UnySupport CPG columns (1 μmole, Glen Research, Sterling, VA). The standard DNA protocol was modified by increasing the coupling time to 360 seconds for FANA phosphoramidites. Cleavage from the solid support and final deprotection of each oligonucleotide was achieved by heating for 18 h at 55° C. in 33% NH4OH. Oligonucleotides were purified by denaturing PAGE, electroeluted, desalted using a YM-3 Centricon centrifugal filter (Millipore, Burlington, MA), and quantified by UV absorbance using a NanoDrop spectrophotometer. FANA containing oligonucleotides were validated by MALDI-TOF mass spectrometry (microflex MALDI-TOF MS, Bruker, Billerica, MA).

In vitro kinetic analysis of RNA cleavage: Kinetic cleavage reactions were performed at 25° C. in 20 μL volumes containing 50 mM Tris-HCl (pH 8.5), 150 mM NaCl, 50 mM MgCl₂, 500 nM Cy5-labeled RNA substrate, 500 nM RNA trigger strand, and 500 nM of each strand of the multicomponent enzyme (Mz-A and Mz-B). The sensor was assembled by heating a solution containing all of the reagents except MgCl₂ for 5 min at 95° C. and cooling for 5 min on ice. Reactions were initiated by the addition of MgCl₂ and left incubating at 25° C. for up to 60 min. Individual time points were collected by diluting 1.5 μL of reaction into 16.5 μL of formamide stop buffer (95% formamide, 25 mM EDTA) and cooling on ice. Samples were denatured for 10 min at 95° C. and analyzed by 15% denaturing urea PAGE. Gels were visualized by the LI-COR Odyssey CLx. k_{obs} values were calculated by fitting cleavage percentage and reaction time (in min) to the first-order decay equation (1) using Prism 8 (GraphPad, San Diego, CA)

$${P}_{t} = {P}_{∞} ( 1 - {e}^{- {k}_{obs} t} ) \qquad ( 1 )$$

Where Pt is the percentage of cleaved substrate at time t, P- is the apparent reaction plateau and k_{obs} is the observed first-order rate constant.

Sensitivity of the 10-23, X10-23, and X10-23 Pro split catalysts: Sensitivity assays were performed at 25° C. in 20 μL volumes containing 50 mM Tris-HCl (pH 8.5), 150 mM NaCl, 50 mM MgCl₂, 500 nM FQ RNA substrate, 500 nM Mz-A, 500 nM Mz-B, and RNA trigger strand. The trigger strand was poised at a range of concentrations to determine the limit of detection. Nuclease-free water was used in the case of the no template control. Mz-A, Mz-B, RNA trigger strand, and FQ RNA substrate were annealed in Tris-HCl and NaCl by heating for 5 min at 95° C. and cooling on ice for 5 min. Reactions were initiated by the addition of MgCl2 and the reaction was monitored by quantitative real-time PCR at 1 min intervals over a period of 2 hours.

RT-RPA preamplification: A lyophilized RPA pellet was resuspended with 29.5 μL rehydration buffer, 1 μL RNase H (5 U/μL), 0.5 μL SuperScript IV RT (200 U/μL), 0.5 μL of forward primer (50 μM), and 0.5 μL of reverse primer (50 μM). A portion (12.8 μL) of the master mix was transferred to each PCR tube. To initiate the assay, 2 μL of magnesium acetate and 6.4 μL of pseudovirus were added to each tube. After brief agitation and centrifugation, the reactions were incubated for 25 min at 42° C. The strip was removed after the first 5 min, briefly vortexed, and placed in a heating device. Then the reaction was inactivated at 95° C. for 5 min. Each RT-RPA tube was placed on ice before split X10-23 Pro detection.

Fluorescence-based detection assay: Split X10-23 Pro detection assays were performed at 37° C. in a 20 μL volume containing 1× T7 RNA polymerase buffer (40 mM Tris-HCl, 6 mM MgCl2, 10 mM DTT, 2 mM spermidine, pH 7.9), 0.5 mM of each NTP, 5 mM DTT, 1.5 U T7 RNA polymerase, 500 nM Mz-A, 500 nM Mz-B, and 500 nM FQ RNA substrate. A portion of the RT-RPA product (2 μL) was transferred to the reaction mixture (18 μL). Reactions were monitored by quantitative real-time PCR at 1 min intervals over a period of 2 hours at 37° C.

Lateral-flow strip detection assay: Split X10-23 Pro detection assays were performed at 25° C. in a 20 μL volume containing 1× T7 RNA polymerase buffer (see above), 0.5 mM of each NTP, 5 mM DTT, 1.5 U T7 RNA polymerase, 500 nM Mz-A, 500 nM Mz-B, and 500 nM F-Biotin RNA substrate. A portion of the RT-RPA product (2 μL) was transferred to the reaction mixture (18 μL). The tubes were then incubated for 1 h at 37° C. before diluting the product in 80 μL HybriDetect assay buffer. After a brief agitation and centrifugation, the HybriDetect lateral flow strips were dipped in the reactions and incubated for 2 min at 25° C. The strips were then imaged, and the bands were quantified using ImageJ (NIH, Bethesda, MD).

Blinded test: 24-IVT RNA pseudovirus samples were prepared with random order by a team member not affiliated with the study. Twelve of the samples contained the SARS-CoV-2 virus poised at concentrations of 20, 50, 100 and 500 aM with 3 replicates, while the remaining samples contain SARS-CoV-1, MERS, rhinovirus, or influenza A, each poised at a concentration of 500 aM with 3 replicates. The REVEALR system was used to identify the SARS-CoV-2. Samples with signal to noise (S/N) ratio >2 would be considered as SARS-CoV-2 positive, or else would be considered as SARS-CoV-2 negative.

Non-limiting examples of oligonucleotides are shown in Table 1.

TABLE 1
List of Oligonucleotides (Note: rA, rG, rC, rU stand for RNA nucleotides. fA, fG, fC, fU
stand for FANA nucleotides.)

| | | SEQ |
|---|---|---|
| Name | Sequence 5′-3′ | ID NO: |
| FluA_fragment | GGCCATGGTGTCTAGGGCCCGGATTGATGCCAGAATTGACTTCGA | 23 |
| | GTCTGGAAGGATTAAGAAGGAAGAGTTCTCTGAGATCATGAAGATC | |
| | TGTTCCACCATTGAAGAACTCAGACGGCAAAAATA | |
| MERS_fragment | ATTGTTACACAATTCGCGCCCGGTACTAAGCTTCCTAAAAACTTCC | 24 |
| | ACATTGAGGGGACTGGAGGCAATAGTCAATCATCTTCAAGAGCCTC | |
| | TAGCTTAA | |
| Rhinovirus_ | GTGTGCTCACTTTGAGTCCTCCGGCCCCTGAATGCGGCTAACCTTA | 25 |
| fragment | AACCTGCAGCCATGGCTCATAAGCCAATGAGTTTATGGTCGTAACG | |
| | AGTAATTGCGGGATGGGACC | |
| SARS-CoV-1 | CCAGCTGGTGGTGCGCTTATAGCTAGGTGTTGGTACCTTCATGAA | 26 |
| fragment | GGCTCAACCAAACTGCTGCATTTAGAGACGTACTTGTTGTTTTAAAT | |
| | AA | |
| SARS-CoV-2_ | AGGTTTCAAACTTTACTTGCTTTACATAGAAGTTATTTGACTCCTGG | 27 |
| fragment | TGATTCTTCTTCAGGTTGGACAGCTGGTGCTGCAGCTTATTATGTG | |
| | GGTTATCTTCAACCTAGGA | |
| Rhinovirus_ | GAAATTAATACGACTCACTATAGGGGTGTGCTCACTTTGAGTCCTC | 28 |
| RPA_FWD | CGGCCCCTG | |
| Rhinovirus_ | GGTCCCATCCCGCAATTACTCGTTACGACC | 29 |
| RPA_RVS | | |
| MERS_FWD | GAAATTAATACGACTCACTATAGGGATTGTTACACAATTCGCGCCC | 30 |
| | GGTACTAAG | |
| MERS_RVS | TTAAGCTAGAGGCTCTTGAAGATGATTGAC | 31 |
| SARS_FWD | GAAATTAATACGACTCACTATAGGGCCAGCTGGTGGTGCGCTTATA | 32 |
| | GCTAGGTGT | |
| SARS_RVS | TTATTTAAAACAACAAGTACGTCTCTAAAT | 33 |
| FluA_FWD | GAAATTAATACGACTCACTATAGGGGGCCATGGTGTCTAGGGCCC | 34 |
| | GGATTGATGC | |
| FluA_RVS | TATTTTTGCCGTCTGAGTTCTTCAATGGTG | 35 |
| SARS-CoV-2-S- | GAAATTAATACGACTCACTATAGGGAGGTTTCAAACTTTACTTGCTT | 36 |
| RPA-FWD | TACATAGA | |
| SARS-CoV-2-S- | TCCTAGGTTGAAGATAACCCACATAATAAG | 37 |
| RPA-RVS | | |
| SARS-CoV_2_ | AGTTATTTGACTCCTGGTGATTCTTCTTCAGGTTGGACAGCTGGTG | 38 |
| Sgene_mid | CTGCAG | |
| S-RPA- | CACCAGCTGTCCAACCTGAAACAACGAGGTTAG | 39 |
| Mz_A_66sub_20 | | |
| S-RPA- | TCATGAGGCTAGCTGAAGAATCACCAGGAGTCAA | 40 |
| Mz_B_66sub_20 | | |
| S-RPA-Mz_A_X10- | CACCAGCTGTCCAACCTGAAACAACGAfGfGfUfUfAfG | 41 |
| 23 | | |
| S-RPA-Mz_B_ | fUfCfAfUfGfAGfGCTAGCfUGAAGAATCACCAGGAGTCAA | 42 |
| X10-23 | | |
| S-RPA-Mz_A_ | fCfAfCfCfAfGfCfUfGfUfCfCfAfAfCfCfUfGfAfAACAACGAfGfGfUfUfAfG | 43 |
| X10-23_Pro | | |
| S-RPA-Mz_B_X10- | fUfCfAfUfGfAGfGCTAGCfUfGfAfAfGfAfAfUfCfAfCfCfAfGfGfAfGfUfCfAf | 44 |
| 23_Pro | A | |
| FBiotin_RNA_ | /56FAM/rCrUrArArCrCrGrUrCrArUrGrA/Bio/ | 45 |
| substrate | | |
| FQ RNA substrate | /56FAM/rCrUrArArCrCrGrUrCrArUrGrA/3IBkFQ/ | 46 |
| Cy5 RNA substrate | /5Cy5/rCrUrArArCrCrGrUrCrArUrGrA | 47 |

### Example 2: REVEALR as a Genotyping Assay

The following is a non-limiting example of the present invention. It is to be understood that said example is not intended to limit the present invention in any way. Equivalents or substitutes are within the scope of the present invention.

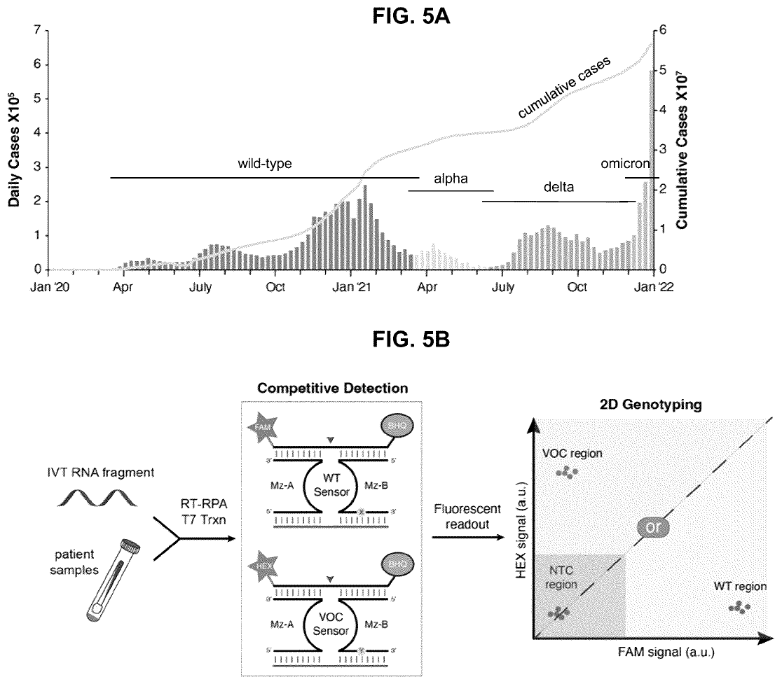

REVEALR is based on a split DNAzyme design strategy in which two halves of a catalytic core (FIG. 5B) self-assemble in the presence of a viral RNA analyte to produce a functionally active catalyst that is capable of cleaving a quenched-fluorescent RNA oligonucleotide strand hybridized to the substrate binding arms of the DNA scaffold. Signal amplification occurs via the multiple turnover activity of the enzyme, as long as the DNAzyme is bound to the viral RNA analyte, and ceases when the complex dissociates into its individual pieces. Previous studies have shown that REVEALR functions with an analytical limit of detection (LoD) of ˜20 aM (˜10 copies/L), which is equivalent to the CRISPR-based methods of SHERLOCK and DETECTR and below the average viral load of COVID-19 patients

Transforming REVEALR into a genotyping assay requires balancing differences in the energetics of hybridization between a perfectly matched viral RNA analyte and a viral analyte carrying a single-nucleotide mutation (i.e., SNP). Since binding to a perfectly matched RNA strand is energetically more favorable than a mismatched strand, properly engineered sensors can be designed to detect a single mutation (transition or transversion) in a nucleic acid sequence. To further enhance the sensitivity of detection, a two-color competitive binding assay was designed that challenges a wild-type and VOC-specific DNAzyme to recognize a genetic mutation within a small region of the viral RNA genome (FIG. 5B). RNA substrates used in the competitive binding assay are equipped with fluorescent dyes (i.e., fluorescein (FAM) and hexachlorofluorescein (HEX)) that have non-overlapping spectral features and nucleic acid sequences that are complementary to their cognate DNAzyme (e.g., wild-type and VOC). Data produced from the competitive assay is visualized in a two-dimensional (2-D) plot (FIG. 5B) with the wild-type analyte producing a strong signal in the lower right quadrant and the VOC analyte generating an equivalently strong signal in the upper left quadrant depending on the identity of the viral RNA analyte present in the sample. By performing the assay against a panel of DNAzymes, each tailored to recognize a specific VOC, it should be possible to unambiguously identify the specific SARS-CoV-2 variant infecting a given patient-derived clinical sample

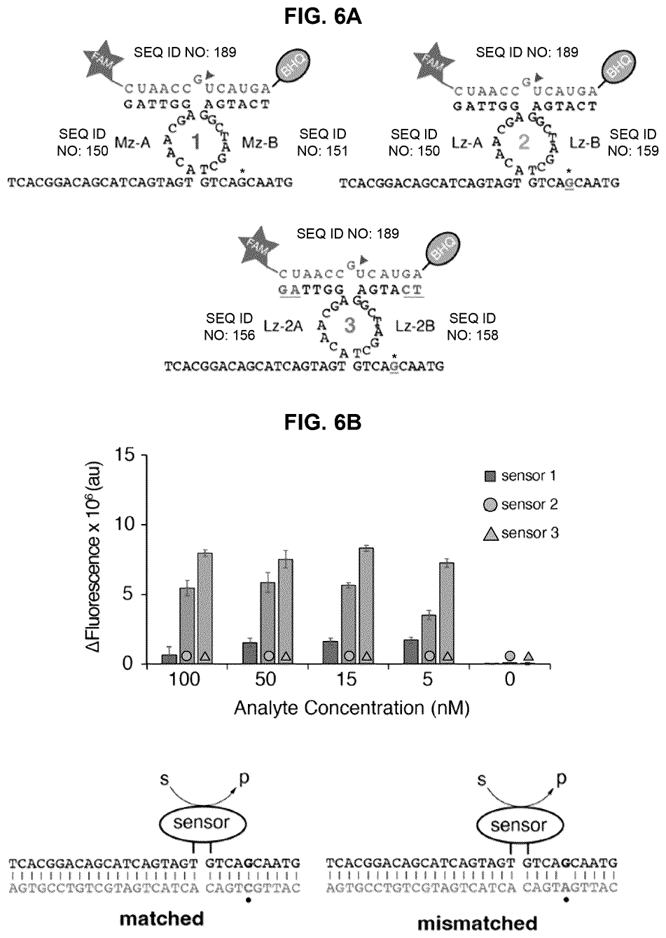

Realizing that chemically modified nucleotides can increase the selectivity of SNP discrimination, the use of locked nucleic acids (LNA) as a chemical tool was explored for improving the activity of the DNAzymes. LNA is a conformationally restricted nucleic acid analog that forces the ribose sugar to adopt a 3′ endo conformation by containing a methylene bridge between the C2′ and C4′ atoms. Thermodynamic studies reveal that LNA increases the melting temperature of DNA oligonucleotides by 4-8° C. per residue when base paired with complementary strands of RNA. Critically, LNA residues enhance the SNP discrimination power of oligonucleotide probes by stabilizing the matched complex to a greater extent than the mismatched complex. In the analysis, DNAzymes carrying LNA residues at both the SNP position and 5′ and 3′ and terminal positions of the substrate binding arms (FIGS. 6A and 6B) function with higher sensitivity than an unmodified version of the DNAzyme or a DNAzyme carrying a single LNA residue at the SNP position. This design configuration was therefore used as the molecular basis of our REVEALR genotyping technology.

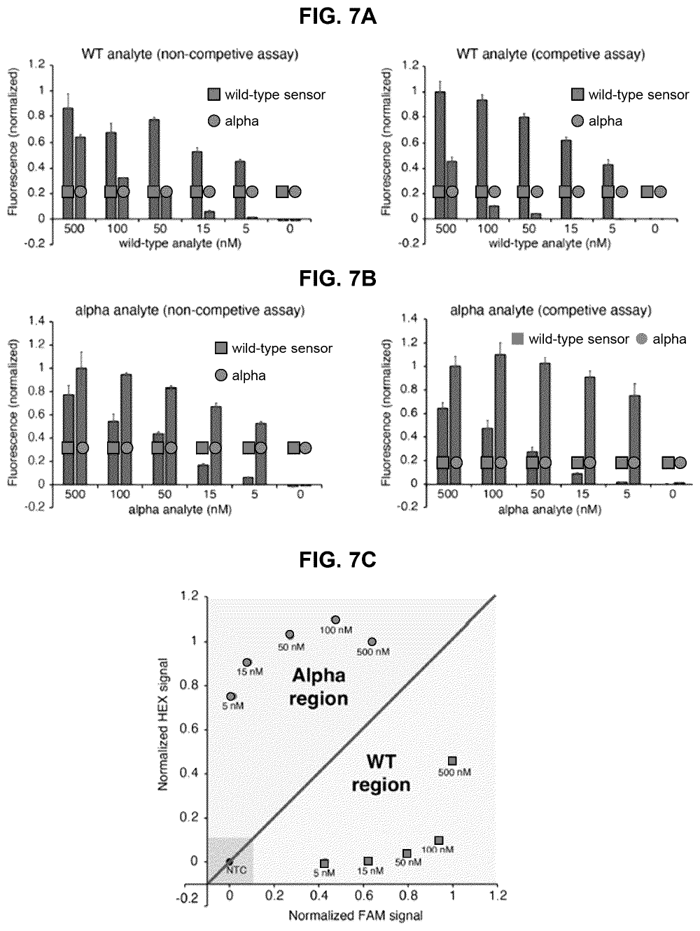

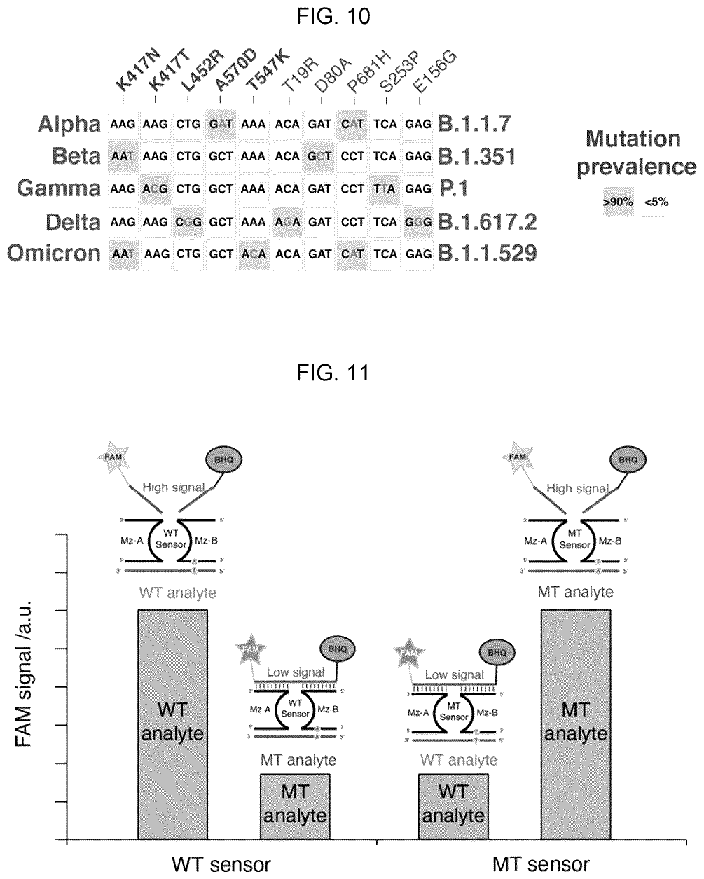

Non-competitive and competitive REVEALR: In designing the REVEALR system, there was initially concern about the potential for cross-reactivity between the DNAzymes. This drawback, which exists for all hybridization-based strategies, could make it difficult to accurately identify VOCs in clinical samples. To evaluate this problem, the cross-reactivity was compared of DNAzymes that were designed to discriminate the wild-type (Wuhan-Hu-1) and alpha (B.1.1.7) strains of SARS-CoV-2 by distinguishing a CA transversion in the viral genome responsible for the A570D mutation in the S1 glycoprotein (FIG. 10, Table 3). The fluorescent signal generated by the wild-type and alpha DNAzyme sensors targeting a perfectly matched DNA analyte or mismatched DNA analyte was measured. SNP detection assays were performed under non-competitive (individual DNAzymes) and competitive (both wild-type and VOC DNAzymes) binding conditions to compare the resolving power of nucleotide discrimination between the two assay formats (FIG. 11). Fluorescence values collected across a range (5-500 nM) of analyte concentrations (FIGS. 7A and 7B), indicate that non-competitive binding conditions allow for accurate SNP detection at low analyte concentrations (5-15 nM range). However, the resolving power of this system is diminished when the analyte concentration reaches a higher level that is more reminiscent of viral RNA samples obtained after isothermal amplification by RT-RPA or LAMP (FIGS. 7A and 7B). This problem is less severe in the competitive binding assay, which exhibits lower background fluorescence due to competition between DNAzymes for the same viral analyte. As an example, the wild-type sensor distinguishes the wild-type analyte (at 100 nM) from the alpha variant by a factor of 10:1 under competitive conditions but only 2:1 under non-competitive conditions. This result suggests that it should be possible to genotype patient samples across a broader range of analyte concentrations, as illustrated in the 2-D plot shown in FIG. 7C.

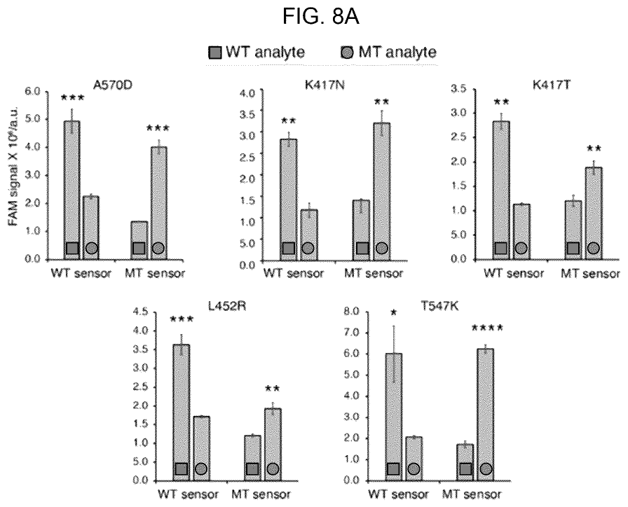

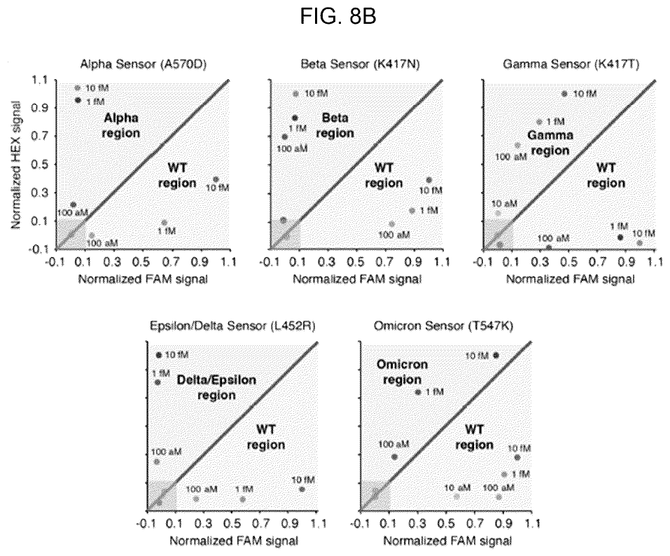

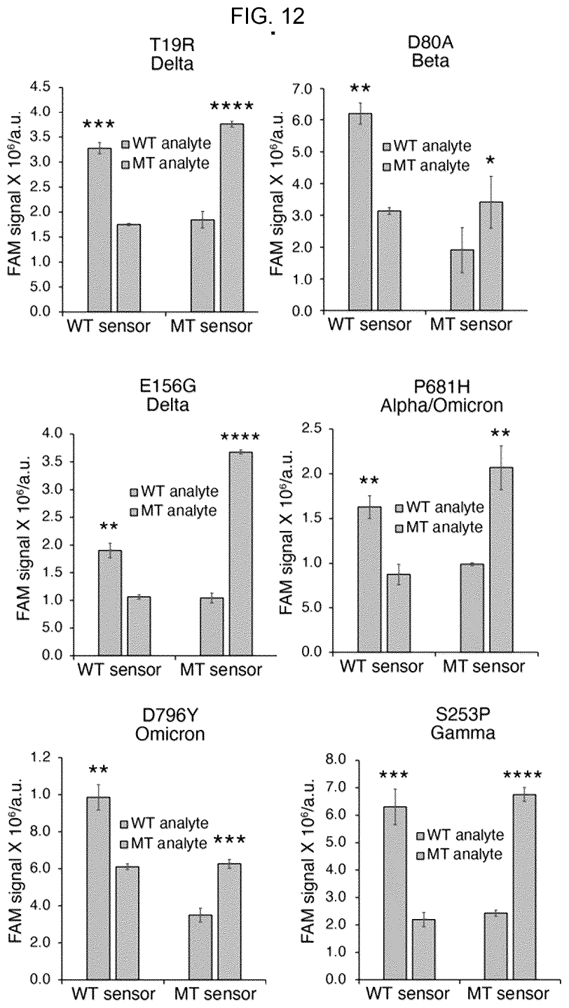

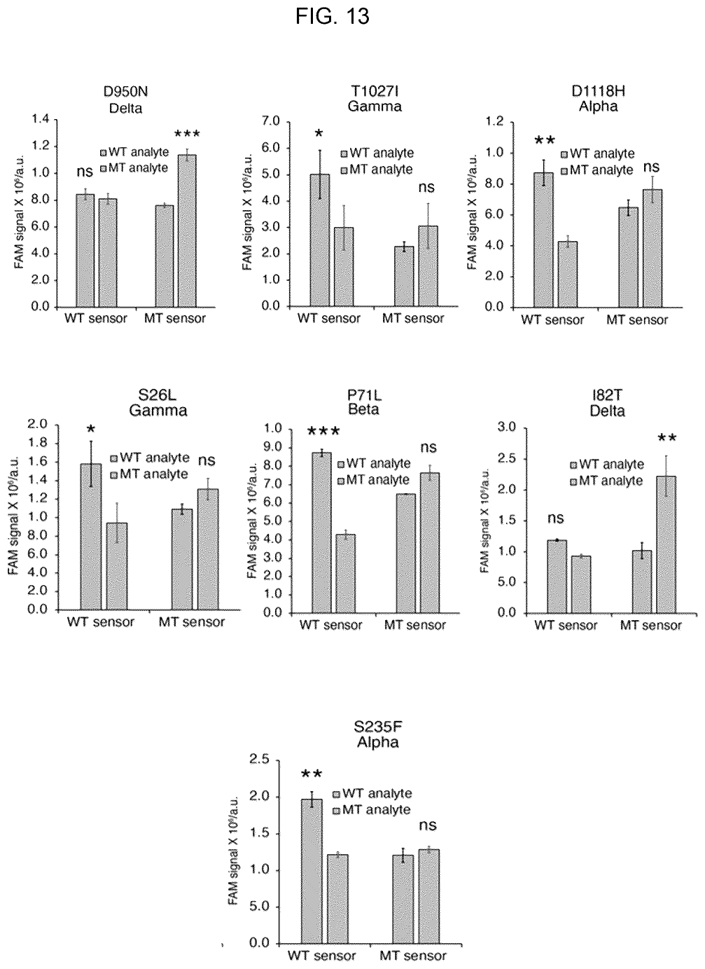

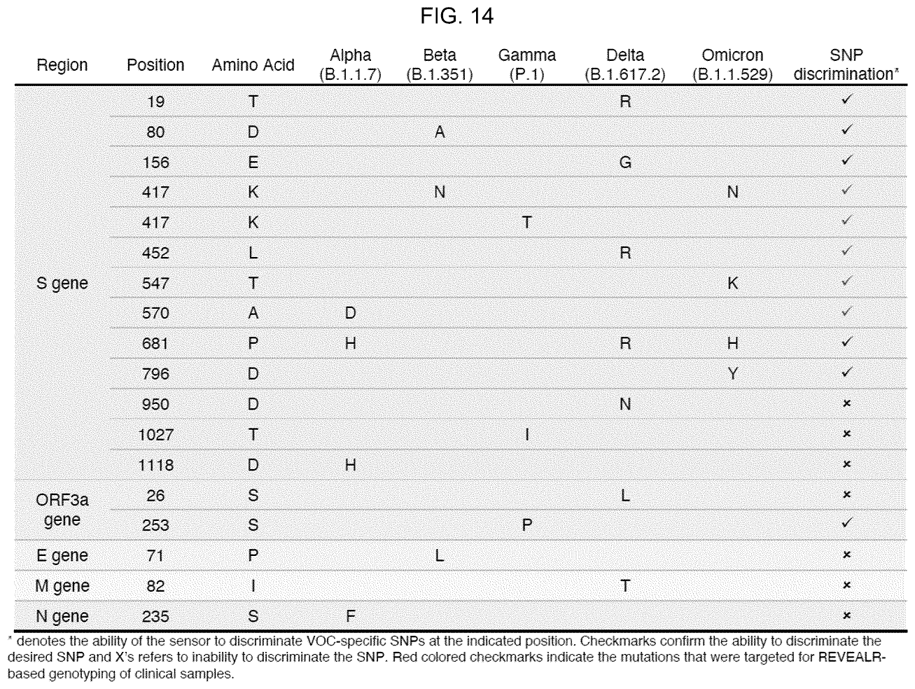

Multicomponent DNAzyme sensors for SARS-CoV-2 Variants of Concern: Eighteen single-nucleotide mutations were evaluated across all regions of the SARS-CoV-2 genome (FIG. 14) to establish a panel of multicomponent DNAzymes that could identify each of the major VOCs observed in the population over the last 24 months. To ensure high sensitivity for each VOC, the analysis was focused on genomic mutations that were prevalent in >90% of each genotypic lineage. The screen identified 11 DNAzymes that functioned with high discriminating power against the wild-type strain in genotyping assays using in vitro transcribed (IVT) RNA analytes. The five most promising sensors (FIG. 8A) are capable of distinguishing the A570D mutation observed in the alpha (B.1.1.7) variant, the K417N mutation observed in the beta (B.1.351) variant, the K417T mutation observed in the gamma (P.1) variant, the L452R mutation observed in the epsilon (B.1.427/9) and delta (B.1.617.2) variants, and the T547K mutation observed in the omicron (B.1.1.529) variant. Another six DNAzymes (FIG. 12) showed strong discrimination against mutations observed in the alpha, beta, gamma, delta, and omicron strains, indicating that these sensors offer an additional layer of sensitivity for future diagnostic assays. The remaining 7 DNAzymes were unable to differentiate the wild-type and mutant analytes (FIG. 13) at this time but could potentially be improved through the use of additional chemical modifications or further optimization of the reaction conditions.

In the context of a REVEALR-based detection assay, where IVT RNA is pre-amplified and detected in a two-step assay, the five most promising sensors were found to function with an analytic LoD of 10-100 aM (FIGS. 8A and 8B). In each case, wild-type and VOC-specific DNAzymes were separately challenged with either the wild-type or mutant analyte in a competitive binding format to assess the detection limit for a simulated viral target. When plotted in the 2-D format (FIG. 8B), wild-type and VOC analytes show strong signal separation along the diagonal axis, illustrating the resolving power of the assay to clearly distinguish wild-type and VOC analytes

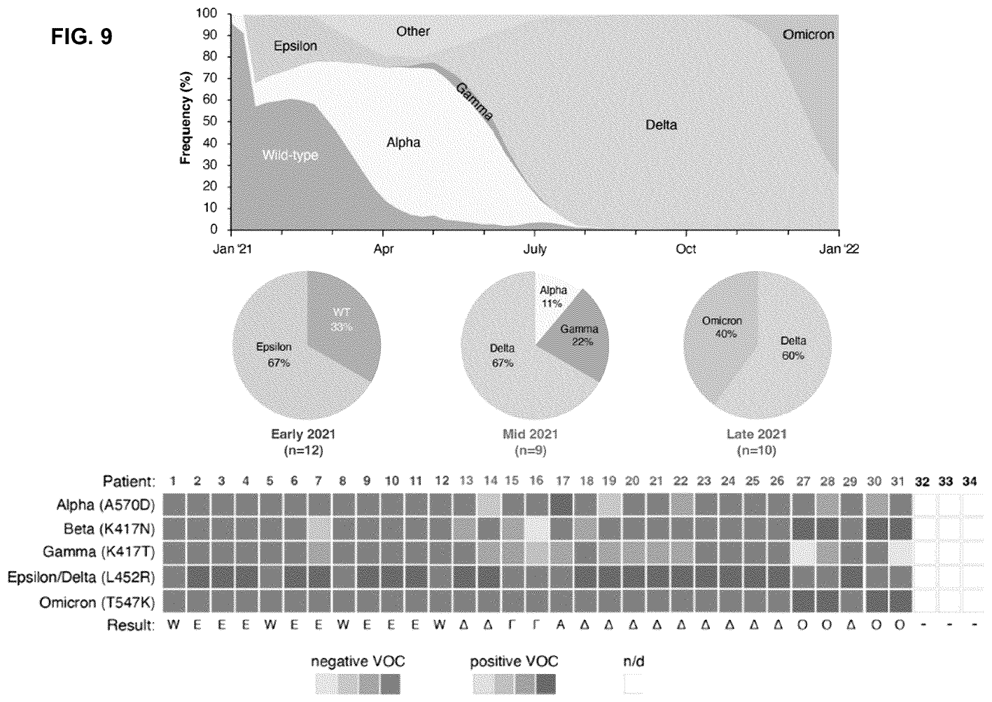

Clinical validation of REVEALR genotyping for SARS-CoV-2 surveillance: Surveillance testing in the United States, both nationally and locally, reveals the spread of SARS-CoV-2 variants of concern across the country. Beginning in January 2021, the country witnessed the chronological rise of five major VOCs, including the alpha (B.1.1.7), gamma (P.1), epsilon (B.1.427/9), delta (B.1.617.2), and omicron (B.1.1.529) strains, along with several other minor variants (FIG. 9). The rapid circulation of these more harmful strains in the public created a need for rapid and accurate genotyping assays that could be deployed as an alternative to traditional sequencing methods, which are slow, costly, and difficult to scale to the population. As a possible solution to this problem, the two-step REVEALR genotyping assay was evaluated on heat-inactivated patient samples collected at the UCI Medical Center in Orange, California. RNA extracted from nasopharyngeal swabs obtained from 34 patients treated in early, mid, and late 2021 were individually assayed in the competitive binding format for the wild-type strain along with the alpha, beta, gamma, epsilon/delta, and omicron variants. Of the 34 samples, 31 derive from COVID positive patients and 3 derive from COVID negative patients (Table 2). All of the samples were sequence verified, either at UCI Medical Center or in our lab on the main campus. REVEALR identified each VOC with perfect accuracy, including patients infected with the wild-type strain, and yielded a clear negative result for each of the three COVID negative swabs (FIG. 9). Importantly, samples collected in early, mid, and late 2021 reflected the abundance of SARS-CoV-2 strains observed locally and nationally at that time, indicating that REVEALR offers a powerful new tool for population surveillance and patient diagnosis. This latter observation could have an important impact on options for therapeutic intervention, as emerging strains, such as delta and omicron, have different virulence levels that can affect patient treatment.

TABLE 2
Summary of patient-derived clinical samples

| | | | | | | | | Varient |
|---|---|---|---|---|---|---|---|---|
| Patient | | | Date | Time | | | | Identification |
| No. | Sex | Age | Collected | frame | Media | CT value | Variant | Source* |
| 1 | M | 55 | Dec. 2, 2020 | Early | HARDY | 12.80 | WT | 2 |
| 2 | F | 58 | Dec. 2, 2020 | Early | REMEL | 17.20 | Epsilon | 1 |
| 3 | M | 69 | Dec. 9, 2020 | Early | HARDY | 3.38 | Epsilon | 1 |
| 4 | M | 70 | Dec. 13, 2020 | Early | HARDY | 13.1 | Epsilon | 2 |
| 5 | M | 46 | Dec. 21, 2020 | Early | REMEL | 17.7 | WT | 2 |
| 6 | F | 30 | Dec. 30, 2020 | Early | HARDY | 8.86 | Epsilon | 2 |
| 7 | F | 68 | Jan. 5, 2021 | Early | HARDY | 11.34 | Epsilon | 1 |
| 8 | F | 30 | Jan. 6, 2021 | Early | REMEL | 12.45 | WT | 2 |
| 9 | M | 23 | Jan. 6, 2021 | Early | HARDY | 4.5 | Epsilon | 2 |
| 10 | F | 64 | Jan. 9, 2021 | Early | REMEL | 16.8 | Epsilon | 2 |
| 11 | F | 36 | Jan. 12, 2021 | Early | REMEL | 14.89 | Epsilon | 2 |
| 12 | F | 26 | Mar. 10, 2021 | Early | REMEL | 17.8 | WT | 2 |
| 13 | M | 39 | Jul. 8, 2021 | Mid | XPERT | 14.83 | Delta | 2 |
| 14 | M | 24 | Jul. 8, 2021 | Mid | XPERT | 16.12 | Delta | 2 |
| 15 | F | 37 | Jul. 15, 2021 | Mid | XPERT | — | Gamma | 1 |
| 16 | F | 14 | Jul. 22, 2021 | Mid | XPERT | — | Gamma | 1 |
| 17 | F | 60 | Jul. 23, 2021 | Mid | XPERT | 12.02 | Alpha | 1 |
| 18 | M | 26 | Jul. 23, 2021 | Mid | XPERT | 14.67 | Delta | 1 |
| 19 | M | 46 | Jul. 26, 2021 | Mid | XPERT | — | Delta | 1 |
| 20 | F | 30 | Jul. 27, 2021 | Mid | XPERT | 17.25 | Delta | 2 |
| 21 | F | 53 | Jul. 28, 2021 | Mid | XPERT | 16.98 | Delta | 1 |
| 22 | F | 35 | Nov. 29, 2021 | Late | BD | 15.15 | Delta | 2 |
| 23 | F | 97 | Dec. 8, 2021 | Late | BD | 8.63 | Delta | 2 |
| 24 | M | 68 | Dec. 8, 2021 | Late | BD | 13.30 | Delta | 2 |
| 25 | M | 31 | Dec. 9, 2021 | Late | BD | 17.80 | Delta | 2 |
| 26 | F | 27 | Dec. 15, 2021 | Late | BD | 9.30 | Delta | 2 |
| 27 | M | 37 | Dec. 20, 2021 | Late | BD | 19.70 | Omicron | 2 |
| 28 | F | 21 | Dec. 22, 2021 | Late | BD | 17.50 | Omicron | 2 |
| 29 | M | 62 | Dec. 22, 2021 | Late | BD | 17.85 | Delta | 2 |
| 30 | M | 69 | Dec. 23, 2021 | Late | BD | 19.05 | Omicron | 2 |
| 31 | F | 59 | Dec. 31, 2021 | Late | — | 17.95 | Omicron | 2 |

*denotes the source of sequence verification of the patient-derived nasopharyngeal samples. Sequence verification of clinical samples was either done by Experimental Tissue Shared Resource Facility team at UCI Medical Center (source 1) or by study researchers via Sanger sequencing (source 2).

Materials: DNA and LNA oligonucleotides were purchased from Integrated DNA Technologies (Coralville, IA). TwistAmp® Basic Kit was purchased from TwistDx (Maidenhead, UK). Solutions of dNTPs (100 mM) were purchased from ThermoFisher (Waltham, MA). T7 RNA polymerase and buffer, as well as M-MuLV Reverse transcriptase, were purchased from Lucigen Corporation (Middleton, WI). HiScribe T7 High Yield RNA Synthesis Kit was purchased from New England Biolabs (Ipswich, MA). GoTaq Probe 1-Step RT-qPCR System was purchased from Promega (Madison, WI).

Multicomponent enzyme screening against 20 genomic positions: Screening experiments were performed in reaction mixtures containing 50 mM Tris-HCl (pH 8.5), 150 mM NaCl, 200 mM MgCl2, 500 nM of each Mz-A and Mz-B (split DNAzymes), 500 nM 7+8 FamQ RNA substrate, and 15 nM of DNA analyte comprising a short segment of either the wild-type or mutant variant. Reactions were performed using each wild-type and mutant DNAzymes targeting both the wild-type and mutant variant, respectively, at each position and monitored by real-time fluorescence using a qRT-PCR instrument at 1 min intervals over a period of 30 min at 37° C. (FIG. 11).

LNA modifications experiments: DNA and LNA versions of the multicomponent DNAzyme (500 nM), named Sensor 1, Sensor 2 and Sensor 3 (FIG. 6A) that target the wild-type region around the A570D mutation observed in the alpha variant, were added to a reaction containing 50 mM Tris-HCl (pH 8.5), 150 mM NaCl, 200 mM MgCl2, 500 nM 6+6 FamQ RNA substrate, and defined concentrations (0-100 nM) of DNA analyte comprising a short segment of either the wild-type or mutant A570D variant. Reactions were monitored by real-time fluorescence using a qRT-PCR instrument at 1 min intervals over a period of 30 min at 25° C. for Sensor 1 and Sensor 2 and 37° C. for Sensor 3.

Non-competitive genotyping experiments: Reaction mixtures were prepared containing 50 mM Tris-HCl (pH 8.5), 150 mM NaCl, 200 mM MgCl2, 500 nM LNA modified split DNAzymes (Sensor 3), 500 nM 6+6 FamQ RNA substrate, and defined concentrations (0-500 nM) of DNA analyte comprising a short segment of either the wild-type or mutant A570D variant. Reactions were monitored by real-time fluorescence using a qRT-PCR instrument at 1 min intervals over a period of 30 min at 37° C.

Competitive genotyping experiments: The reactions were performed as described for the non-competitive genotyping experiments, with the exception of the solution containing LNA modified split DNAzymes (Sensor 3) that target both the wild-type and mutant analyte. To distinguish the signal generated from the wild-type and mutant analytes, the quenched RNA substrates were prepared with non-overlapping fluorescent dyes. Fluorescein (FAM) was used for the wild-type sensor and hexachlorofluorescein (HEX) was used for the mutant sensor. Additionally, the RNA substrates carried unique sequences that were complementary to their specific LNA modified split DNAzymes (Sensor 3). Reactions were monitored by real-time fluorescence using a qRT-PCR instrument at 1 min intervals over a period of 30 min at 37° C.

In vitro transcribed RNA: RNA analytes mimicking specific mutations (K417N/T, L452R, T547K, A570D) in the SARS-CoV-2 genome were prepared by in vitro transcription (IVT). IVT reactions were performed using the HiScribe T7 High Yield RNA Synthesis Kit. Each reaction contained 10 mM of each NTP, 1× reaction buffer, 3 μL PCR product, 2 μL T7 RNA polymerase mixture, and 5 μL nuclease free water. Reactions were incubated at 37° C. overnight. Crude RNA was purified by 15% denaturing urea PAGE and electroeluted, either under 180 V for 3 h or 60 V overnight. Purified RNA was desalted with an Amicon Ultra 0.5 mL 30 k centrifugal filter from EMD Millipore (Burlington, MA), quantified by NanoDrop, and diluted in nuclease-free water to desired concentrations.

REVEALR-based genotyping: A lyophilized RPA pellet was resuspended with 29.5 μL rehydration buffer, 1 μL M-MuLV-RT (200 U/μL), 0.5 μL forward primer (50 μM), 0.5 μL reverse primer (50 μM), and 1.25 uL ATP (100 mM). A portion (13.1 μL) of the master mix was transferred to the reaction tube. To initiate the assay, 2 μL magnesium acetate and 4.9 μL of IVT RNA or purified RNA from clinical samples were added to the side of each tube, without contacting the master mix. After briefly vortexing to mix the magnesium acetate initiator into the reaction, and subsequent centrifugation, the reactions were incubated for 25 min at 42° C. The reaction tube was removed after the first 5 min, briefly vortexed, and returned to the heating device. After incubation, the reaction was inactivated by heating the reaction tube for 5 min at 95° C. The RT-RPA reactions were then placed on ice before T7 transcription.

The dsDNA produced by RT-RPA was forward transcribed into ssRNA by in vitro transcription. T7 transcription reactions contained 1× T7 RNA Pol Buffer, 0.5 mM NTPs, 30 U T7 RNA polymerase, and 2 uL RT-RPA product for a 20 uL total volume. Reactions were incubated for 1 h at 37° C. before being used for the competitive REVEALR genotyping assay.

The competitive genotyping assay was performed as described above, except for one step in which the reactions were seeded with 6 uL of in vitro transcribed RNA or purified RNA obtained from clinical samples that was amplified by RT-RPA/T7 isothermal amplification instead of DNA segments. Reactions were monitored by real-time fluorescence using a qRT-PCR instrument at 1 min intervals over a period of 1 h at 37° C.

Sensitivity test and data normalization: The sensitivity test was performed with 10 fM, 1 fM, 100 aM, and 10 aM of in vitro transcribed wild-type and mutant RNA following the competitive REVEALR genotyping assay described above. Data was normalized using the no template control (NTC) FAM/HEX signals as the negative value, 10 fM wild-type FAM signals as FAM positive value, and 10 fM mutant HEX signals as HEX positive value. The functions are listed as follows.

$$Normalized FAM signal = \frac{( real FAM signal - negative FAM signal )}{( positive FAM signal - negative FAM signal )} \\ Normalized HEX signal = \frac{( real HEX signal - negative HEX signal )}{( positive HEX signal - negative HEX signal )}$$

Evaluation of patient-derived clinical samples: Nasopharyngeal swabs from 34 patients were obtained from the COVID-19 Research Biobank of the Experimental Tissue Shared Resource Facility at University of California, Irvine. Each sample was collected and heat inactivated for 1 h at 80° C. by trained medical professionals at the University of California Medical Center in Orange, California. The samples were collected from patients treated in early, mid, and late 2021 at UCI Medical Center. The variant types of 9 samples were identified by the hospital and the other 25 samples were identified via Sanger sequencing. SARS-CoV 2 viral RNA samples were purified following the CDC recommended Qiagen DSP Viral RNA Mini kit protocol. The REVEALR genotyping system was used to detect the extracted RNA viral RNA using fluorescence readout as described above.

Sanger sequencing: Clinical samples were amplified using the GoTaq Probe 1-Step RT-qPCR System to target regions of interest. RT-PCR was performed following the manufacturer's recommended protocol. dsDNA products were purified with an agarose gel purification step using 2% agarose gels. The DNA was extracted from the gel using the Monarch DNA Gel Extraction Kit from New England Biolabs (Ipswich, MA), and cleaned-up using the DNA Clean and Concentrator Kit from Zymo Research (Irvine, CA).

Non-limiting examples of oligonucleotides are shown in Table 3.

TABLE 3
List of Oligonucleotides (Note: rA, rG, rC, rU stand for RNA nucleotides. fA, fG, fC, fU
stand for FANA nucleotides, IA, IG, IC, IU stand for LNA nucleotides; Black stands for DNA
nucleotides; Bolded Nucleotides reflect the SNP site on the sensor; Bolded/Underlined
Nucleotides reflect the SNP site on the analyte; italicized nucleotides signified the T7
promoter region; Underlined Nucleotides_represent the substrate binding arm; lower case
nucleotides stands for the catalytic core of the DNAzyme).

| Name | Sequence 5′-3′ | SEQ ID NO: |
|---|---|---|
| RNA Substrates | | |
| FamQ-RNA-sub_6 + 6 | /56-FAM/rCrUrArArCrCrGrUrCrArUrGrA/3IABKFQ/ | 48 |
| HexQ-RNA-sub-6 + 6 | /5HEX/rUrUrCrCrUrCrGrUrCrCrCrUrG/3BHQ_1/ | 49 |
| FamQ-RNA-sub-7 + 8 | /56FAM/rCrUrUrUrCrCrUrCrGrUrCrCrCrUrGrG/3IABKFQ/ | 50 |
| T19R | | |
| T19R_WT_analyte | TCTTACAACCAGAACTCAATTACCCCCTGC | 51 |
| T19R_MT_analyte | TCTTAGAACCAGAACTCAATTACCCCCTGC | 52 |
| Mz-A_T19R-7 + 8sub | GCAGGGGGTAATTGAGTTCTacaacgaGAGGAAAG | 53 |
| Mz-B_T19R-WT-7 + 8sub | CCAGGGAggctagctGGTTGTAAGA | 54 |
| Mz-B_T19R-MT-7 + 8sub | CCAGGGAggctagctGGTTCTAAGA | 55 |
| S26L | | |
| S26L_WT_analyte | TCCTTCAGATTTTGTTCGCGCTACTGCAAC | 56 |
| S26L_MT_analyte | TCCTTTAGATTTTGTTCGCGCTACTGCAAC | 57 |
| Mz-A_S26L-7 + 8sub | GTTGCAGTAGCGCGAACAAAacaacgaGAGGAAAG | 58 |
| Mz-B_S26L-WT-7 + 8sub | CCAGGGAggctagctATCTGAAGGA | 59 |
| Mz-B_S26L-MT-7 + 8sub | CCAGGGAggctagctATCTAAAGGA | 60 |
| P71L | | |
| P71L_WT_analyte | AGTTCCTGATCTTCTGGTCTAAACGAACTA | 61 |
| P71L_MT_analyte | AGTTCTTGATCTTCTGGTCTAAACGAACTA | 62 |
| Mz-A_P71L-7 + 8su | TAGTTCGTTTAGACCAGAAGacaacgaGAGGAAAG | 63 |
| Mz-B_P71L-WT-7 + 8sub | CCAGGGAggctagctATCAGGAACT | 64 |
| Mz-B_P71L-MT-7 + 8sub | CCAGGGAggctagctATCAAGAACT | 65 |
| D80A | | |
| D80A_WT_analyte | GTTTGATAACCCTGTCCTACCATTTAATGA | 66 |
| D80A_MT_analyte | GTTTGCTAACCCTGTCCTACCATTTAATGA | 67 |
| Mz-A_D80A-7 + 8sub | TCATTAAATGGTAGGACAGGacaacgaGAGGAAAG | 68 |
| Mz-B_D80A-WT-7 + 8sub | CCAGGGAggctagctGTTATCAAAC | 69 |
| Mz-B_D80A-MT-7 + 8sub | CCAGGGAggctagctGTTAGCAAAC | 70 |
| 182T | | |
| 182T_WT_analyte | TGCTATCGCAATGGCTTGTCTTGTAGGCTT | 71 |
| 182T_MT_analyte | TGCTACCGCAATGGCTTGTCTTGTAGGCTT | 72 |
| Mz-A_182T-7 + 8sub | AAGCCTACAAGACAAGCCATacaacgaGAGGAAAG | 73 |
| Mz-B_182T-WT-7 + 8sub | CCAGGGAggctagctTGCGATAGCA | 74 |
| Mz-B_182T-MT-7 + 8sub | CCAGGGAggctagctTGCGGTAGCA | 75 |
| E156G | | |
| E156G_WT_analyte | AAGTGAGTTCAGAGTTTATTCTAGTGCGAA | 76 |
| E156G_MT_analyte | AAGTGGGTTCAGAGTTTATTCTAGTGCGAA | 77 |
| Mz-A_E156G-7 + 8sub | TTCGCACTAGAATAAACTCTacaacgaGAGGAAAG | 78 |
| Mz-B_E156G-WT-7 + 8sub | CCAGGGAggctagctGAACTCACTT | 79 |
| Mz-B_E156G-MT-7 + 8sub | CCAGGGAggctagctGAACCCACTT | 80 |
| S235F | | |
| S235F_WT_analyte | AATGTCTGGTAAAGGCCAACAACAACAAGG | 81 |
| S235F_MT_analyte | AATGTTTGGTAAAGGCCAACAACAACAAGG | 82 |
| Mz-A_S235F-7 + 8sub | CCTTGTTGTTGTTGGCCTTTacaacgaGAGGAAAG | 83 |
| Mz-B_S235F-WT-7 + 8sub | CCAGGGAggctagctACCAGACATT | 84 |
| Mz-B_S235F-MT-7 + 8sub | CCAGGGAggctagctACCAAACATT | 85 |
| S253P | | |
| S253P_WT_analyte | GTTCATCCGGAGTTGTTAATCCAGTAATGG | 86 |
| S253P_MT_analyte | GTTCACCCGGAGTTGTTAATCCAGTAATGG | 87 |
| Mz-A_S253P-7 + 8sub | CCATTACTGGATTAACAACTacaacgaGAGGAAAG | 88 |
| Mz-B_S253P-WT-7 + 8sub | CCAGGGAggctagctCCGGATGAAC | 89 |
| Mz-B_S253P-MT-7 + 8sub | CCAGGGAggctagctCCGGGTGAAC | 90 |
| K417N/T | | |
| K417_WT_analyte | GGAAAGATTGCTGATTATAATTATAAATTA | 91 |
| K417N_MT_analyte | GGAAATATTGCTGATTATAATTATAAATTA | 92 |
| K417T_MT_analyte | GGAACGATTGCTGATTATAATTATAAATTA | 93 |
| K417_WT_RPA_template | GAAGTCAGACAAATCGCTCCAGGGCAAACTGGAAAG | 94 |
| | ATTGCTGATTATAATTATAAATTACCAGATGATTTTAC | |
| | AGGCTGCGTTATAGCT | |
| K417N_MT_RPA_template | GAAGTCAGACAAATCGCTCCAGGGCAAACTGGAAAT | 95 |
| | ATTGCTGATTATAATTATAAATTACCAGATGATTTTAC | |
| | AGGCTGCGTTATAGCT | |
| K417T_MT_RPA_template | GAAGTCAGACAAATCGCTCCAGGGCAAACTGGAACG | 96 |
| | ATTGCTGATTATAATTATAAATTACCAGATGATTTTAC | |
| | AGGCTGCGTTATAGCT | |
| K417_RPA_FWD | GAAATTAATACGACTCACTATAGGGGAAATTAATACG | 97 |
| | ACTCACTATAGGGGTGATGAAGTCAGACAAATCGCT | |
| | CCAGGGC | |
| K417_RPA_RVS | TTCCAAGCTATAACGCAGCCTGTAAAATCA | 98 |
| Mz-A_K417-7 + 8sub | TAATTTATAATTATAATCAGacaacgaGAGGAAAG | 99 |
| Mz-B_K417N-WT-7 + 8sub | CCAGGGAggctagctCAATCTTTCC | 100 |
| Mz-B_K417N-MT-7 + 8sub | CCAGGGAggctagctCAATATTTCC | 101 |
| Mz-B_K417T-MT-7 + 8sub | CCAGGGAggctagctCAATCGTTCC | 102 |
| Lz-B_K417N-MT- | ITICATGAggctagctCAATIATTTCC | 103 |
| 6 + 6sub_FAM | | |
| Lz-B_K417T-MT- | ITICATGAggctagctCAATCIGTTCC | 104 |
| 6 + 6sub_FAM | | |
| Lz-A_K417-6 + 6sub_FAM | TAATTTATAATTATAATCAGacaacgaGGTTIAIG | 105 |
| Lz-A_K417-6 + 6sub_HEX | TAATTTATAATTATAATCAGacaacgaGAGGIAIA | 106 |
| Lz-B_K417N-MT-6 + 6sub_ | ICIAGGGAggctagctCAATIATTTCC | 107 |
| HEX | | |
| Lz-B_K417T-MT-6 + 6sub_ | ICIAGGGAggctagctCAATCIGTTCC | 108 |
| HEX | | |
| Lz-B_K417N-MT-6 + 6sub_ | CAGGGAggctagctCAATIATTTCC | 109 |
| HEX | | |
| Lz-B_K417T-MT-6 + 6sub_ | CAGGGAggctagctCAATCIGTTCC | 110 |
| HEX | | |
| Lz-B_K417-WT-FAM | ITICATGAggctagctCAATICTTTCC | 111 |
| Lz-B_K417-MT-HEX | ICIAGGGAggctagctCAATIATTTCC | 112 |
| L452R | | |
| L452R_WT_analyte | TTACCTGTATAGATTGTTTAGGAAGTCTAA | 113 |
| L452R_MT_analyte | TTACCGGTATAGATTGTTTAGGAAGTCTAA | 114 |
| L452R_WT_template | AATTCTAACAATCTTGATTCTAAGGTTGGTGGTAATTA | 115 |
| | TAATTACCTGTATAGATTGTTTAGGAAGTCTAATCTCA | |
| | AACCTTTTGAGAGA | |
| L452R_MT_template | AATTCTAACAATCTTGATTCTAAGGTTGGTGGTAATTA | 116 |
| | TAATTACCGGTATAGATTGTTTAGGAAGTCTAATCTC | |
| | AAACCTTTTGAGAGA | |
| RPA_L452R_RVS | ATATCTCTCTCAAAAGGTTTGAGATTAGAC | 117 |
| RPA_L452R_FWD | GAAATTAATACGACTCACTATAGGGCTTGGAATTCTA | 118 |
| | ACAA TCTTGATTCTAAGG | |
| Mz-A_L452R-7 + 8sub | TTAGACTTCCTAAACAATCTacaacgaGAGGAAAG | 119 |
| Mz-B_L452R-WT-7 + 8sub | CCAGGGAggctagctATACAGGTAA | 120 |
| Mz-B_L452R-MT-7 + 8sub | CCAGGGAggctagctATACCGGTAA | 121 |
| Lz-A_L452R-HEX | TTAGACTTCCTAAACAATCTacaacgaGAGGIAIA | 122 |
| Lz-B_L452R-WT-HEX | ICIAGGGAggctagctATACIAGGTAA | 123 |
| Lz-B_L452R-MT-HEX | ICIAGGGAggctagctATACICGGTAA | 124 |
| Lz-A_L452R-FAM | TTAGACTTCCTAAACAATCTacaacgaGGTTIAIG | 125 |
| Lz-B_L452R-WT-FAM | ITICATGAggctagctATACIAGGTAA | 126 |
| Lz-B_L452R-MT-FAM | ITICATGAggctagctATACICGGTAA | 127 |
| T547K | | |
| T547K_WT_template | AATTTGGTTAAAAACAAATGTGTCAATTTCAACTTCAA | 128 |
| | TGGTTTAACAGGCACAGGTGTTCTTACTGAGTCTAAC | |
| | AAAAAGTT TCTGCCT | |
| T547K_MT_template | AATTTGGTTAAAAACAAATGTGTCAATTTCAACTTCAA | 129 |
| | TGGTTTAAAAGGCACAGGTGTTCTTACTGAGTCTAAC | |
| | AAAAAGTTTCTGCCT | |
| RPA_T547K_FWD | GAAATTAATACGACTCACTATAGGGCTACTAATTTGG | 130 |
| | TTAAAAACAAATGTGTCA | |
| RPA_T547K_RVS | TGGAAAGGCAGAAACTTTTTGTTAGACTCA | 131 |
| Mz-A_T547K-7 + 8sub | GACTCAGTAAGAACACCTGTacaacgaGAGGAAAG | 132 |
| Mz-B_T547K-WT-7 + 8sub | CCAGGGAggctagctGCCTGTTAAA | 133 |
| Mz-B_T547K-MT-7 + 8sub | CCAGGGAggctagctGCCTTTTAAA | 134 |
| Lz-A_T547K-FAM | GACTCAGTAAGAACACCTGTacaacgaGGTTIAIG | 135 |
| Lz-B_T547K-WT-FAM | ITICATGAggctagctGCCTIGTTAAA | 136 |
| Lz-B_T547K-MT-FAM | ITICATGAggctagctGCCTITTTAAA | 137 |
| LZ-A_T547K-HEX | GACTCAGTAAGAACACCTGTacaacgaGAGGIAIA | 138 |
| Lz-B_T547K-WT-HEX | ICIAGGGAggctagctGCCTIGTTAAA | 139 |
| Lz-B_T547K-MT-HEX | ICIAGGGAggctagctGCCTITTTAAA | 140 |
| A570D | | |
| A570D_WT_analyte_S | CATTGCTGACACTACTGATGCTGTCCGTGA | 141 |
| A570D_MT_analyte_S | CATTGATGACACTACTGATGCTGTCCGTGA | 142 |
| A570D_WT_template | TCTAACAAAAAGTTTCTGCCTTTCCAACAATTTGGCA | 143 |
| | GAGACATTGCTGACACTACTGATGCTGTCCGTGATC | |
| | CACAGACACTTGAGATT | |
| A570D_MT_template | TCTAACAAAAAGTTTCTGCCTTTCCAACAATTTGGCA | 144 |
| | GAGACATTGATGACACTACTGATGCTGTCCGTGATC | |
| | CACAGACACTTGAGATT | |
| RPA_A570D_RVS | TCAAGAATCTCAAGTGTCTGTGGATCACGG | 145 |
| RPA_A570D_FWD | GAAATTAATACGACTCACTATAGGGCTGAGTCTAACA | 146 |
| | AAAAGTTTCTGCCTTTCC | |
| Mz-A_A570D-7 + 8sub | TCACGGACAGCATCAGTAGTacaacgaGAGGAAAG | 147 |
| Mz-B_A570D-WT-7 + 8sub | CCAGGGAggctagctGTCAGCAATG | 148 |
| Mz-B_A570D-MT-7 + 8sub | CCAGGGAggctagctGTCATCAATG | 149 |
| Mz-66_A_A570D-FAM | TCACGGACAGCATCAGTAGTacaacgaGGTTAG | 150 |
| Mz-66_B_A570D-WT-FAM | TCATGAggctagctGTCAGCAATG | 151 |
| Mz-66_B_A570D-MT-FAM | TCATGAggctagctGTCATCAATG | 152 |
| Lz-A_A570D-HEX | TCACGGACAGCATCAGTAGTacaacgaGAGGIAIA | 153 |
| Lz-B_A570D-WT-HEX | ICIAGGGAggctagctGTCAIGCAATG | 154 |
| Lz-B_A570D-MT-HEX | ICIAGGGAggctagctGTCAITCAATG | 155 |
| Lz-A_A570D-FAM | TCACGGACAGCATCAGTAGTacaacgaGGTTIAIG | 156 |
| Lz-B_A570D-WT-FAM | ITICATGAggctagctGTCAIGCAATG | 157 |
| Lz-B_A570D-MT-FAM | ITICATGAggctagctGTCAITCAATG | 158 |
| Lz-B_A570D-WT- | TCATGAggctagctGTCAIGCAATG | 159 |
| FAM_SNP | | |
| Lz-B_A570D-MT- | TCATGAggctagctGTCAITCAATG | 160 |
| FAM_SNP | | |
| P681H | | |
| P681_WT_analyte | TTCTCCTCGGCGGGCACGTAGTGTAGCTAG | 161 |
| P681H_MT_analyte | TTCTCATCGGCGGGCACGTAGTGTAGCTAG | 162 |
| Mz-A_P681H-7 + 8sub | CTAGCTACACTACGTGCCCGacaacgaGAGGAAAG | 163 |
| Mz-B_P681H-WT-7 + 8sub | CCAGGGAggctagctCCGAGGAGAA | 164 |
| Mz-B_P681H-MT-7 + 8sub | CCAGGGAggctagctCCGATGAGAA | 165 |
| D796Y | | |
| D796Y_WT_analyte | TTAAAGATTTTGGTGGTTTTAATTTTTCAC | 166 |
| D796Y_MT_analyte | TTAAATATTTTGGTGGTTTTAATTTTTCAC | 167 |
| Mz-A_D796Y-7 + 8sub | GTGAAAAATTAAAACCACCAacaacgaGAGGAAAG | 168 |
| Mz-B_D796Y-WT-7 + 8sub | CCAGGGAggctagctAAATCTTTAA | 169 |
| Mz-B_D796Y-MT-7 + 8sub | CCAGGGAggctagctAAATATTTAA | 170 |
| D950N | | |
| D950N_WT_analyte | TTCAAGATGTGGTCAACCAAAATGCACAAG | 171 |
| D950N_MT_analyte | TTCAAAATGTGGTCAACCAAAATGCACAAG | 172 |
| Mz-A_D950N-7 + 8sub | CTTGTGCATTTTGGTTGACCacaacgaGAGGAAAG | 173 |
| Mz-B_D950N-WT-7 + 8sub | CCAGGGAggctagctACATCTTGAA | 174 |
| Mz-B_D950N-MT-7 + 8sub | CCAGGGAggctagctACATTTTGAA | 175 |
| T10271 | | |
| T1027\|_WT_analyte | TGCTACTAAAATGTCAGAGTGTGTACTTGG | 176 |
| T1027\|_MT_analyte | TGCTATTAAAATGTCAGAGTGTGTACTTGG | 177 |
| Mz-A_T10271-7 + 8sub | CCAAGTACACACTCTGACATacaacgaGAGGAAAG | 178 |
| Mz-B_T1027I-WT-7 + 8sub | CCAGGGAggctagctTTTAGTAGCA | 179 |
| Mz-B_T1027I-MT-7 + 8sub | CCAGGGAggctagctTTTAATAGCA | 180 |
| D1118H | | |
| D1118H_WT_analyte | CTACAGACAACACATTTGTGTCTGGTAACT | 181 |
| D1118H_MT_analyte | CTACACACAACACATTTGTGTCTGGTAACT | 182 |
| Mz-A_D1118H-7 + 8sub | AGTTACCAGACACAAATGTGacaacgaGAGGAAAG | 183 |
| Mz-B_D1118H-WT-7 + 8sub | CCAGGGAggctagctTTGTCTGTAG | 184 |
| Mz-B_D1118H-MT-7 + 8sub | CCAGGGAggctagctTTGTGTGTAG | 185 |

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A multicomponent nucleic acid enzyme composition comprising:
a. a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end; and
b. a second nucleic enzyme component comprising a second nucleic acid catalytic core accordingly to SEQ ID NO: 2 (5′-3′ GGCTACGU), SEQ ID NO: 3 (5′-3′ GGCTACGT) wherein the residue of SEQ ID NO: 3 at position 8 is a nucleic acid analog, or SEQ ID NO: 4 (5′-3′ GGCTAGCT) flanked by a second nucleic acid substrate binding arm at its 5′ end and a second nucleic acid trigger arm at its 3′ end, wherein at least 10% of residues of the second nucleic acid trigger arm are nucleic acid analogues;
wherein upon assembly of the first nucleic acid catalytic core and second nucleic acid catalytic core, the first nucleic acid catalytic core and second nucleic acid catalytic core form an active enzyme for cleaving nucleic acid.

2.-6. (canceled)

7. A multicomponent nucleic acid enzyme composition comprising:
a. a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end, and
b. a second nucleic enzyme component comprising a second nucleic acid catalytic core according to SEQ ID NO: 2 (5′-3′ GGCTACGU), SEQ ID NO: 3 (5′-3′ GGCTACGT), or SEQ ID NO: 4 (5′-3′ GGCTAGCT) flanked by a second nucleic acid substrate binding arm at its 5′ end and a second nucleic acid trigger arm at its 3′ end, wherein the residues of SEQ ID NO: 2, SEQ ID NO: 3, or SEQ ID NO: 4 at position 2 or 8 are nucleic acid analogues;
wherein upon assembly of the first nucleic acid catalytic core and second nucleic acid catalytic core, the first nucleic acid catalytic core and second nucleic acid catalytic core form an active enzyme for cleaving nucleic acid.

8. (canceled)

9. The composition of claim 7, wherein the nucleic acid analogue is selected from: 2′-fluoroarabino nucleic acid (FANA), locked nucleic acid (LNA), peptide nucleic acid (PNA), hexose nucleic acid (HNA), threose nucleic acid (TNA), cyclohexenyl nucleic acid (CeNA), morpholino nucleic acid (MNA), a 2′ substituted RNA, and a sugar modified analog.

10. The composition of claim 9, wherein the 2′ substituted RNA is (OCH3), 2′ amino (NH2), or 2′ methoxyethoxy (MOE): wherein the sugar modified analog is Me-ANA, MOE-ANA, or 6′-methyl F-HNA.

11. (canceled)

12. The composition of claim 7, wherein the composition assembles into the active enzyme in the presence of a trigger sequence.

13. The composition of claim 12, wherein the trigger sequence is a nucleic acid; wherein the nucleic acid is RNA, DNA, or a combination thereof.

14. (canceled)

15. The composition of claim 12, wherein the trigger sequence comprises a nucleic acid having a particular sequence, wherein the trigger arms are complementary to the trigger sequence.

16. (canceled)

17. The composition of claim 7, wherein the composition is for detecting nucleic acid of a pathogen; wherein the pathogen is a virus, bacterium, a fungus, a protozoan, or a parasite.

18. (canceled)

19. The composition of claim 7, wherein the composition is for genotyping.

20. (canceled)

21. The composition of claim 12, wherein the active enzyme can cleave a nucleic acid reporter upon detection of the trigger sequence.

22. The composition of claim 21, wherein the nucleic acid reporter comprises an oligonucleotide having a cleavage site, a label disposed on one side of the cleavage site and a masking molecule disposed on one side of the cleavage site opposite the label, wherein the masking molecule prevents the label from being detectable when the nucleic acid reporter is not cleaved, and the label is detectable when the nucleic acid reporter is cleaved at its cleavage site; wherein cleaving the nucleic acid reporter generates a detectable signal.

23. (canceled)

24. The composition of claim 7, wherein the composition can be engineered to detect a specific trigger sequence.

25. The composition of claim 7, wherein the composition can be engineered to detect a specific nucleic acid reporter.

26. The composition of claim 7, wherein the composition is capable of analyte detection of <20 aM.

27.-23. (canceled)

29. The composition of claim 7, wherein at least 10% at least 25% at least 50% at least 75% at least 90% or at least 95% of the residues of the trigger arms and substrate binding arms are nucleic acid analogs.

30.-33. (canceled)

34. The composition of claim 7, wherein all of the residues of the trigger arms and substrate binding arms are nucleic acid analogs.

35.-36. (canceled)

37. The composition of claim 7, wherein the first substrate binding arm and the second substrate binding arm are 5 to 15 nucleotides in length.

33. The composition of claim 7, wherein the first trigger arm and second trigger arm are at least 6 to 15 nucleotides in length.

39.-52. (canceled)

53. A kit comprising:
a. a multicomponent enzyme composition comprising:
i. a first nucleic enzyme component comprising a first nucleic acid catalytic core according to SEQ ID NO: 1 (5′-3′ ACAACGA) flanked by a first nucleic acid substrate binding arm at its 3′ end and a first nucleic acid trigger arm at its 5′ end; and
ii. a second nucleic enzyme component comprising a second nucleic acid catalytic core accordingly to SEQ ID NO: 2 (5′-3′ GGCTACGU) SEQ ID NO: 3 (5′-3′ GGCTACGT) wherein the residue of SEQ ID NO: 3 at position 3 is a nucleic acid analog, or SEQ ID NO: 4 (5-3′ GGCTAGCT) flanked by a second nucleic acid substrate binding arm at its 5′ end and a second nucleic acid trigger arm at its 3′ end, wherein at least 10% of residues of the second nucleic acid trigger arm are nucleic acid analogues;
wherein upon assembly of the first nucleic acid catalytic core and second nucleic acid catalytic core, the first nucleic acid catalytic core and second nucleic acid catalytic core form an active enzyme for cleaving nucleic acid; and
b. a nucleic acid reporter comprising an oligonucleotide having a cleavage site, a label disposed on one side of the cleavage site and a masking molecule disposed on one side of the cleavage site opposite the label, wherein the masking molecule prevents the label from being detectable when the nucleic acid reporter is not cleaved and the label is detectable when the nucleic acid reporter is cleaved at its cleavage site wherein cleaving the nucleic acid reporter generates a detectable signal.

54. The kit of claim 53, wherein the enzyme composition becomes active upon detection of a trigger sequence, the nucleic acid reporter is cleaved when the enzyme composition becomes active, and the label of the nucleic acid reporter becomes detectable when the nucleic acid reporter is cleaved.

55.-88. (canceled)


SEQUENCE LISTING (189 sequences)

SEQ ID NO: 1 - DNA, 7 bp, Artificial Sequence
  Catalytic Core
acaacga

SEQ ID NO: 2 - DNA, 8 bp, Artificial Sequence
  Catalytic Core
ggctacgu

SEQ ID NO: 3 - DNA, 8 bp, Artificial Sequence
  Catalytic Core
ggctacgt

SEQ ID NO: 4 - DNA, 8 bp, Artificial Sequence
  Catalytic Core
ggctagct

SEQ ID NO: 5 - DNA, 8 bp, Artificial Sequence
  Catalytic Core
  misc_feature (2)..(2): Nucleic Acid Analogue
ggctacgu

SEQ ID NO: 6 - DNA, 8 bp, Artificial Sequence
  Catalytic Core
  misc_feature (2)..(2): Nucleic Acid Analogue
ggctacgt

SEQ ID NO: 7 - DNA, 8 bp, Artificial Sequence
  Catalytic Core
  misc_feature (2)..(2): Nucleic Acid Analogue
ggctagct

SEQ ID NO: 8 - DNA, 8 bp, Artificial Sequence
  Catalytic Core
  misc_feature (8)..(8): Nucleic Acid Analogue
ggctacgu

SEQ ID NO: 9 - DNA, 8 bp, Artificial Sequence
  Catalytic Core
  misc_feature (8)..(8): Nucleic Acid Analogue
ggctacgt

SEQ ID NO: 10 - DNA, 8 bp, Artificial Sequence
  Catalytic Core
  misc_feature (8)..(8): Nucleic Acid Analogue
ggctagct

SEQ ID NO: 11 - DNA, 8 bp, Artificial Sequence
  Catalytic Core
  misc_feature (2)..(2): Nucleic Acid Analogue
  misc_feature (8)..(8): Nucleic Acid Analogue
ggctacgu

SEQ ID NO: 12 - DNA, 8 bp, Artificial Sequence
  Catalytic Core
  misc_feature (2)..(2): Nucleic Acid Analogue
  misc_feature (8)..(8): Nucleic Acid Analogue
ggctacgt

SEQ ID NO: 13 - DNA, 8 bp, Artificial Sequence
  Catalytic Core
  misc_feature (2)..(2): Nucleic Acid Analogue
  misc_feature (8)..(8): Nucleic Acid Analogue
ggctagct

SEQ ID NO: 14 - DNA, 8 bp, Artificial Sequence
  Catalytic Core
  misc_feature (2)..(2): 2′-fluoroarabino nucleic acid (FANA) residue
ggctacgu

SEQ ID NO: 15 - DNA, 8 bp, Artificial Sequence
  Catalytic Core
  misc_feature (2)..(2): 2′-fluoroarabino nucleic acid (FANA) residue
ggctacgt

SEQ ID NO: 16 - DNA, 8 bp, Artificial Sequence
  Catalytic Core
  misc_feature (2)..(2): 2′-fluoroarabino nucleic acid (FANA) residue
ggctagct

SEQ ID NO: 17 - DNA, 8 bp, Artificial Sequence
  Catalytic Core
  misc_feature (8)..(8): 2′-fluoroarabino nucleic acid (FANA) residue
ggctacgu

SEQ ID NO: 18 - DNA, 8 bp, Artificial Sequence
  Catalytic Core
  misc_feature (8)..(8): 2′-fluoroarabino nucleic acid (FANA) residue
ggctacgt

SEQ ID NO: 19 - DNA, 8 bp, Artificial Sequence
  Catalytic Core
  misc_feature (8)..(8): 2′-fluoroarabino nucleic acid (FANA) residue
ggctagct

SEQ ID NO: 20 - DNA, 8 bp, Artificial Sequence
  Catalytic Core
  misc_feature (2)..(2): 2′-fluoroarabino nucleic acid (FANA) residue
  misc_feature (8)..(8): 2′-fluoroarabino nucleic acid (FANA) residue
ggctacgu

SEQ ID NO: 21 - DNA, 8 bp, Artificial Sequence
  Catalytic Core
  misc_feature (2)..(2): 2′-fluoroarabino nucleic acid (FANA) residue
  misc_feature (8)..(8): 2′-fluoroarabino nucleic acid (FANA) residue
ggctacgt

SEQ ID NO: 22 - DNA, 8 bp, Artificial Sequence
  Catalytic Core
  misc_feature (2)..(2): 2′-fluoroarabino nucleic acid (FANA) residue
  misc_feature (8)..(8): 2′-fluoroarabino nucleic acid (FANA) residue
ggctagct

SEQ ID NO: 23 - DNA, 126 bp, Artificial Sequence
  FluA_fragment
ggccatggtgtctagggcccggattgatgccagaattgacttcgagtctggaaggattaagaaggaagagttctctgagatcatgaagatctgttccaccattgaagaactcagacggcaaaaata

SEQ ID NO: 24 - DNA, 100 bp, Artificial Sequence
  MERS_fragment
attgttacacaattcgcgcccggtactaagcttcctaaaaacttccacattgaggggactggaggcaatagtcaatcatcttcaagagcctctagcttaa

SEQ ID NO: 25 - DNA, 112 bp, Artificial Sequence
  Rhinovirus_fragment
gtgtgctcactttgagtcctccggcccctgaatgcggctaaccttaaacctgcagccatggctcataagccaatgagtttatggtcgtaacgagtaattgcgggatgggacc

SEQ ID NO: 26 - DNA, 94 bp, Artificial Sequence
  SARS-CoV-1_fragment
ccagctggtggtgcgcttatagctaggtgttggtaccttcatgaaggctcaaccaaactgctgcatttagagacgtacttgttgttttaaataa

SEQ ID NO: 27 - DNA, 112 bp, Artificial Sequence
  SARS-CoV-2_fragment
aggtttcaaactttacttgctttacatagaagttatttgactcctggtgattcttcttcaggttggacagctggtgctgcagcttattatgtgggttatcttcaacctagga

SEQ ID NO: 28 - DNA, 55 bp, Artificial Sequence
  Rhinovirus_RPA_FWD
gaaattaatacgactcactataggggtgtgctcactttgagtcctccggcccctg

SEQ ID NO: 29 - DNA, 30 bp, Artificial Sequence
  Rhinovirus_ RPA_RVS
ggtcccatcccgcaattactcgttacgacc

SEQ ID NO: 30 - DNA, 55 bp, Artificial Sequence
  MERS_FWD
gaaattaatacgactcactatagggattgttacacaattcgcgcccggtactaag

SEQ ID NO: 31 - DNA, 30 bp, Artificial Sequence
  MERS_RVS
ttaagctagaggctcttgaagatgattgac

SEQ ID NO: 32 - DNA, 55 bp, Artificial Sequence
  SARS_FWD
gaaattaatacgactcactatagggccagctggtggtgcgcttatagctaggtgt

SEQ ID NO: 33 - DNA, 30 bp, Artificial Sequence
  SARS_RVS
ttatttaaaacaacaagtacgtctctaaat

SEQ ID NO: 34 - DNA, 55 bp, Artificial Sequence
  FluA_FWD
gaaattaatacgactcactatagggggccatggtgtctagggcccggattgatgc

SEQ ID NO: 35 - DNA, 30 bp, Artificial Sequence
  FluA_RVS
tatttttgccgtctgagttcttcaatggtg

SEQ ID NO: 36 - DNA, 55 bp, Artificial Sequence
  SARS-CoV-2-S-RPA-FWD
gaaattaatacgactcactatagggaggtttcaaactttacttgctttacataga

SEQ ID NO: 37 - DNA, 30 bp, Artificial Sequence
  SARS-CoV-2-S-RPA-RVS
tcctaggttgaagataacccacataataag

SEQ ID NO: 38 - DNA, 52 bp, Artificial Sequence
  SARS-CoV_2_ Sgene_mid
agttatttgactcctggtgattcttcttcaggttggacagctggtgctgcag

SEQ ID NO: 39 - DNA, 33 bp, Artificial Sequence
  S-RPA-Mz_A_66sub_20
caccagctgtccaacctgaaacaacgaggttag

SEQ ID NO: 40 - DNA, 34 bp, Artificial Sequence
  S-RPA-Mz_B_66sub_20
tcatgaggctagctgaagaatcaccaggagtcaa

SEQ ID NO: 41 - DNA, 33 bp, Artificial Sequence
  S-RPA-Mz_A_X10-23
  misc_feature (28)..(33): 2′-fluoroarabino nucleic acid (FANA) residues
caccagctgtccaacctgaaacaacgagguuag

SEQ ID NO: 42 - DNA, 34 bp, Artificial Sequence
  S-RPA-Mz_B_ X10-23
  misc_feature (1)..(6): 2′-fluoroarabino nucleic acid (FANA) residue
  misc_feature (8)..(8): 2′-fluoroarabino nucleic acid (FANA) residue
  misc_feature (14)..(14): 2′-fluoroarabino nucleic acid (FANA) residue
ucaugaggctagcugaagaatcaccaggagtcaa

SEQ ID NO: 43 - DNA, 33 bp, Artificial Sequence
  S-RPA-Mz_A_ X10-23_Pro
  misc_feature (1)..(20): 2′-fluoroarabino nucleic acid (FANA) residue
  misc_feature (28)..(33): 2′-fluoroarabino nucleic acid (FANA) residue
caccagcuguccaaccugaaacaacgagguuag

SEQ ID NO: 44 - DNA, 34 bp, Artificial Sequence
  S-RPA-Mz_B_X10-23_Pro
  misc_feature (1)..(6): 2′-fluoroarabino nucleic acid (FANA) residue
  misc_feature (8)..(8): 2′-fluoroarabino nucleic acid (FANA) residue
  misc_feature (14)..(34): 2′-fluoroarabino nucleic acid (FANA) residue
ucaugaggctagcugaagaaucaccaggagucaa

SEQ ID NO: 45 - RNA, 13 nt, Artificial Sequence
  FBiotin_RNA_ substrate
cuaaccgucauga

SEQ ID NO: 46 - RNA, 13 nt, Artificial Sequence
  FQ RNA substrate
cuaaccgucauga

SEQ ID NO: 47 - RNA, 13 nt, Artificial Sequence
  Cy5 RNA substrate
cuaaccgucauga

SEQ ID NO: 48 - RNA, 13 nt, Artificial Sequence
  FamQ-RNA-sub_6+6
cuaaccgucauga

SEQ ID NO: 49 - RNA, 13 nt, Artificial Sequence
  HexQ-RNA-sub-6+6
uuccucgucccug

SEQ ID NO: 50 - RNA, 16 nt, Artificial Sequence
  FamQ-RNA-sub-7+8
cuuuccucgucccugg

SEQ ID NO: 51 - DNA, 30 bp, Artificial Sequence
  T19R_WT_analyte
tcttacaaccagaactcaattaccccctgc

SEQ ID NO: 52 - DNA, 30 bp, Artificial Sequence
  T19R_MT_analyte
tcttagaaccagaactcaattaccccctgc

SEQ ID NO: 53 - DNA, 35 bp, Artificial Sequence
  Mz-A_T19R-7+8sub
gcagggggtaattgagttctacaacgagaggaaag

SEQ ID NO: 54 - DNA, 25 bp, Artificial Sequence
  Mz-B_T19R-WT-7+8sub
ccagggaggctagctggttgtaaga

SEQ ID NO: 55 - DNA, 25 bp, Artificial Sequence
  Mz-B_T19R-MT-7+8sub
ccagggaggctagctggttctaaga

SEQ ID NO: 56 - DNA, 30 bp, Artificial Sequence
  S26L_WT_analyte
tccttcagattttgttcgcgctactgcaac

SEQ ID NO: 57 - DNA, 30 bp, Artificial Sequence
  S26L_MT_analyte
tcctttagattttgttcgcgctactgcaac

SEQ ID NO: 58 - DNA, 35 bp, Artificial Sequence
  Mz-A_S26L-7+8sub
gttgcagtagcgcgaacaaaacaacgagaggaaag

SEQ ID NO: 59 - DNA, 25 bp, Artificial Sequence
  Mz-B_S26L-WT-7+8sub
ccagggaggctagctatctgaagga

SEQ ID NO: 60 - DNA, 25 bp, Artificial Sequence
  Mz-B_S26L-MT-7+8sub
ccagggaggctagctatctaaagga

SEQ ID NO: 61 - DNA, 30 bp, Artificial Sequence
  P71L_WT_analyte
agttcctgatcttctggtctaaacgaacta

SEQ ID NO: 62 - DNA, 30 bp, Artificial Sequence
  P71L_MT_analyte
agttcttgatcttctggtctaaacgaacta

SEQ ID NO: 63 - DNA, 35 bp, Artificial Sequence
  Mz-A_P71L-7+8su
tagttcgtttagaccagaagacaacgagaggaaag

SEQ ID NO: 64 - DNA, 25 bp, Artificial Sequence
  Mz-B_P71L-WT-7+8sub
ccagggaggctagctatcaggaact

SEQ ID NO: 65 - DNA, 25 bp, Artificial Sequence
  Mz-B_P71L-MT-7+8sub
ccagggaggctagctatcaagaact

SEQ ID NO: 66 - DNA, 30 bp, Artificial Sequence
  D80A_WT_analyte
gtttgataaccctgtcctaccatttaatga

SEQ ID NO: 67 - DNA, 30 bp, Artificial Sequence
  D80A_MT_analyte
gtttgctaaccctgtcctaccatttaatga

SEQ ID NO: 68 - DNA, 35 bp, Artificial Sequence
  Mz-A_D80A-7+8sub
tcattaaatggtaggacaggacaacgagaggaaag

SEQ ID NO: 69 - DNA, 25 bp, Artificial Sequence
  Mz-B_D80A-WT-7+8sub
ccagggaggctagctgttatcaaac

SEQ ID NO: 70 - DNA, 25 bp, Artificial Sequence
  Mz-B_D80A-MT-7+8sub
ccagggaggctagctgttagcaaac

SEQ ID NO: 71 - DNA, 30 bp, Artificial Sequence
  I82T_WT_analyte
tgctatcgcaatggcttgtcttgtaggctt

SEQ ID NO: 72 - DNA, 30 bp, Artificial Sequence
  I82T_MT_analyte
tgctaccgcaatggcttgtcttgtaggctt

SEQ ID NO: 73 - DNA, 35 bp, Artificial Sequence
  Mz-A_I82T-7+8sub
aagcctacaagacaagccatacaacgagaggaaag

SEQ ID NO: 74 - DNA, 25 bp, Artificial Sequence
  Mz-B_I82T-WT-7+8sub
ccagggaggctagcttgcgatagca

SEQ ID NO: 75 - DNA, 25 bp, Artificial Sequence
  Mz-B_I82T-MT-7+8sub
ccagggaggctagcttgcggtagca

SEQ ID NO: 76 - DNA, 30 bp, Artificial Sequence
  E156G_WT_analyte
aagtgagttcagagtttattctagtgcgaa

SEQ ID NO: 77 - DNA, 30 bp, Artificial Sequence
  E156G_MT_analyte
aagtgggttcagagtttattctagtgcgaa

SEQ ID NO: 78 - DNA, 35 bp, Artificial Sequence
  Mz-A_E156G-7+8sub
ttcgcactagaataaactctacaacgagaggaaag

SEQ ID NO: 79 - DNA, 25 bp, Artificial Sequence
  Mz-B_E156G-WT-7+8sub
ccagggaggctagctgaactcactt

SEQ ID NO: 80 - DNA, 25 bp, Artificial Sequence
  Mz-B_E156G-MT-7+8sub
ccagggaggctagctgaacccactt

SEQ ID NO: 81 - DNA, 30 bp, Artificial Sequence
  S235F_WT_analyte
aatgtctggtaaaggccaacaacaacaagg

SEQ ID NO: 82 - DNA, 30 bp, Artificial Sequence
  S235F_MT_analyte
aatgtttggtaaaggccaacaacaacaagg

SEQ ID NO: 83 - DNA, 35 bp, Artificial Sequence
  Mz-A_S235F-7+8sub
ccttgttgttgttggcctttacaacgagaggaaag

SEQ ID NO: 84 - DNA, 25 bp, Artificial Sequence
  Mz-B_S235F-WT-7+8sub
ccagggaggctagctaccagacatt

SEQ ID NO: 85 - DNA, 25 bp, Artificial Sequence
  Mz-B_S235F-MT-7+8sub
ccagggaggctagctaccaaacatt

SEQ ID NO: 86 - DNA, 30 bp, Artificial Sequence
  S253P_WT_analyte
gttcatccggagttgttaatccagtaatgg

SEQ ID NO: 87 - DNA, 30 bp, Artificial Sequence
  S253P_MT_analyte
gttcacccggagttgttaatccagtaatgg

SEQ ID NO: 88 - DNA, 35 bp, Artificial Sequence
  Mz-A_S253P-7+8sub
ccattactggattaacaactacaacgagaggaaag

SEQ ID NO: 89 - DNA, 25 bp, Artificial Sequence
  Mz-B_S253P-WT-7+8sub
ccagggaggctagctccggatgaac

SEQ ID NO: 90 - DNA, 25 bp, Artificial Sequence
  Mz-B_S253P-MT-7+8sub
ccagggaggctagctccgggtgaac

SEQ ID NO: 91 - DNA, 30 bp, Artificial Sequence
  K417_WT_analyte
ggaaagattgctgattataattataaatta

SEQ ID NO: 92 - DNA, 30 bp, Artificial Sequence
  K417N_MT_analyte
ggaaatattgctgattataattataaatta

SEQ ID NO: 93 - DNA, 30 bp, Artificial Sequence
  K417T_MT_analyte
ggaacgattgctgattataattataaatta

SEQ ID NO: 94 - DNA, 90 bp, Artificial Sequence
  K417_WT_RPA_template
gaagtcagacaaatcgctccagggcaaactggaaagattgctgattataattataaattaccagatgattttacaggctgcgttatagct

SEQ ID NO: 95 - DNA, 90 bp, Artificial Sequence
  K417N_MT_RPA_template
gaagtcagacaaatcgctccagggcaaactggaaatattgctgattataattataaattaccagatgattttacaggctgcgttatagct

SEQ ID NO: 96 - DNA, 90 bp, Artificial Sequence
  K417T_MT_RPA_template
gaagtcagacaaatcgctccagggcaaactggaacgattgctgattataattataaattaccagatgattttacaggctgcgttatagct

SEQ ID NO: 97 - DNA, 80 bp, Artificial Sequence
  K417_RPA_FWD
  misc_feature (1)..(23): T7 promoter
gaaattaatacgactcactataggggaaattaatacgactcactataggggtgatgaagtcagacaaatcgctccagggc

SEQ ID NO: 98 - DNA, 30 bp, Artificial Sequence
  K417_RPA_RVS
ttccaagctataacgcagcctgtaaaatca

SEQ ID NO: 99 - DNA, 35 bp, Artificial Sequence
  Mz-A_K417-7+8sub
taatttataattataatcagacaacgagaggaaag

SEQ ID NO: 100 - DNA, 25 bp, Artificial Sequence
  Mz-B_K417N-WT-7+8sub
ccagggaggctagctcaatctttcc

SEQ ID NO: 101 - DNA, 25 bp, Artificial Sequence
  Mz-B_K417N-MT-7+8sub
ccagggaggctagctcaatatttcc

SEQ ID NO: 102 - DNA, 25 bp, Artificial Sequence
  Mz-B_K417T-MT-7+8sub
ccagggaggctagctcaatcgttcc

SEQ ID NO: 103 - DNA, 24 bp, Artificial Sequence
  Lz-B_K417N-MT-6+6sub_FAM
  misc_feature (1)..(2): Locked Nucleic Acid (LNA) Analogue
  misc_feature (19)..(19): Locked Nucleic Acid (LNA) & SNP site on the analyte
tcatgaggctagctcaatatttcc

SEQ ID NO: 104 - DNA, 24 bp, Artificial Sequence
  Lz-B_K417T-MT-6+6sub_FAM
  misc_feature (1)..(2): Locked Nucleic Acid (LNA) Analogue
  misc_feature (20)..(20): Locked Nucleic Acid (LNA) Analogue & SNP site on the analyte
tcatgaggctagctcaatcgttcc

SEQ ID NO: 105 - DNA, 33 bp, Artificial Sequence
  Lz-A_K417-6+6sub_FAM
  misc_feature (32)..(33): Locked Nucleic Acid (LNA) Analogue
taatttataattataatcagacaacgaggttag

SEQ ID NO: 106 - DNA, 33 bp, Artificial Sequence
  Lz-A_K417-6+6sub_HEX
  misc_feature (32)..(33): Locked Nucleic Acid (LNA) Analogue
taatttataattataatcagacaacgagaggaa

SEQ ID NO: 107 - DNA, 24 bp, Artificial Sequence
  Lz-B_K417N-MT-6+6sub_ HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctcaatatttcc

SEQ ID NO: 108 - DNA, 24 bp, Artificial Sequence
  Lz-B_K417T-MT-6+6sub_ HEX
  misc_feature (1)..(2): Locked Nucleic Acid (LNA) Analogue
  misc_feature (20)..(20): Locked Nucleic Acid (LNA) Analogue & SNP site on the analyte
cagggaggctagctcaatcgttcc

SEQ ID NO: 109 - DNA, 24 bp, Artificial Sequence
  Lz-B_K417N-MT-6+6sub_HEX
  misc_feature (19)..(19): Locked Nucleic Acid (LNA) Analogue & SNP site on the analyte
cagggaggctagctcaatatttcc

SEQ ID NO: 110 - DNA, 24 bp, Artificial Sequence
  Lz-B_K417T-MT-6+6sub_ HEX
  misc_feature (20)..(20): Locked Nucleic Acid (LNA) Analogue & SNP site on the analyte
cagggaggctagctcaatcgttcc

SEQ ID NO: 111 - DNA, 24 bp, Artificial Sequence
  Lz-B_K417-WT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctcaatctttcc

SEQ ID NO: 112 - DNA, 24 bp, Artificial Sequence
  Lz-B_K417-MT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctcaatatttcc

SEQ ID NO: 113 - DNA, 30 bp, Artificial Sequence
  L452R_WT_analyte
ttacctgtatagattgtttaggaagtctaa

SEQ ID NO: 114 - DNA, 30 bp, Artificial Sequence
  L452R_MT_analyte
ttaccggtatagattgtttaggaagtctaa

SEQ ID NO: 115 - DNA, 90 bp, Artificial Sequence
  L452R_WT_template
aattctaacaatcttgattctaaggttggtggtaattataattacctgtatagattgtttaggaagtctaatctcaaaccttttgagaga

SEQ ID NO: 116 - DNA, 90 bp, Artificial Sequence
  L452R_MT_template
aattctaacaatcttgattctaaggttggtggtaattataattaccggtatagattgtttaggaagtctaatctcaaaccttttgagaga

SEQ ID NO: 117 - DNA, 30 bp, Artificial Sequence
  RPA_L452R_RVS
atatctctctcaaaaggtttgagattagac

SEQ ID NO: 118 - DNA, 55 bp, Artificial Sequence
  RPA_L452R_FWD
  misc_feature (1)..(22): T7 Promoter
gaaattaatacgactcactatagggcttggaattctaacaatcttgattctaagg

SEQ ID NO: 119 - DNA, 35 bp, Artificial Sequence
  Mz-A_L452R-7+8sub
ttagacttcctaaacaatctacaacgagaggaaag

SEQ ID NO: 120 - DNA, 25 bp, Artificial Sequence
  Mz-B_L452R-WT-7+8sub
ccagggaggctagctatacaggtaa

SEQ ID NO: 121 - DNA, 25 bp, Artificial Sequence
  Mz-B_L452R-MT-7+8sub
ccagggaggctagctataccggtaa

SEQ ID NO: 122 - DNA, 33 bp, Artificial Sequence
  Lz-A_L452R-HEX
  misc_feature (32)..(33): Locked Nucleic Acid (LNA) Analogue
ttagacttcctaaacaatctacaacgagaggaa

SEQ ID NO: 123 - DNA, 24 bp, Artificial Sequence
  Lz-B_L452R-WT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctatacaggtaa

SEQ ID NO: 124 - DNA, 24 bp, Artificial Sequence
  Lz-B_L452R-MT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctataccggtaa

SEQ ID NO: 125 - DNA, 33 bp, Artificial Sequence
  Lz-A_L452R-FAM
  misc_feature (32)..(33): Locked Nucleic Acid (LNA) Analogue
ttagacttcctaaacaatctacaacgaggttag

SEQ ID NO: 126 - DNA, 24 bp, Artificial Sequence
  Lz-B_L452R-WT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctatacaggtaa

SEQ ID NO: 127 - DNA, 24 bp, Artificial Sequence
  Lz-B_L452R-MT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctataccggtaa

SEQ ID NO: 128 - DNA, 90 bp, Artificial Sequence
  T547K_WT_template
aatttggttaaaaacaaatgtgtcaatttcaacttcaatggtttaacaggcacaggtgttcttactgagtctaacaaaaagtttctgcct

SEQ ID NO: 129 - DNA, 90 bp, Artificial Sequence
  T547K_MT_template
aatttggttaaaaacaaatgtgtcaatttcaacttcaatggtttaaaaggcacaggtgttcttactgagtctaacaaaaagtttctgcct

SEQ ID NO: 130 - DNA, 55 bp, Artificial Sequence
  RPA_T547K_FWD
  misc_feature (1)..(22): T7 Promoter
gaaattaatacgactcactatagggctactaatttggttaaaaacaaatgtgtca

SEQ ID NO: 131 - DNA, 30 bp, Artificial Sequence
  RPA_T547K_RVS
tggaaaggcagaaactttttgttagactca

SEQ ID NO: 132 - DNA, 35 bp, Artificial Sequence
  Mz-A_T547K-7+8sub
gactcagtaagaacacctgtacaacgagaggaaag

SEQ ID NO: 133 - DNA, 25 bp, Artificial Sequence
  Mz-B_T547K-WT-7+8sub
ccagggaggctagctgcctgttaaa

SEQ ID NO: 134 - DNA, 25 bp, Artificial Sequence
  Mz-B_T547K-MT-7+8sub
ccagggaggctagctgccttttaaa

SEQ ID NO: 135 - DNA, 33 bp, Artificial Sequence
  Lz-A_T547K-FAM
  misc_feature (32)..(33): Locked Nucleic Acid (LNA) Analogue
gactcagtaagaacacctgtacaacgaggttag

SEQ ID NO: 136 - DNA, 24 bp, Artificial Sequence
  Lz-B_T547K-WT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctgcctgttaaa

SEQ ID NO: 137 - DNA, 24 bp, Artificial Sequence
  Lz-B_T547K-MT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctgccttttaaa

SEQ ID NO: 138 - DNA, 33 bp, Artificial Sequence
  Lz-A_T547K-HEX
  misc_feature (32)..(33): Locked Nucleic Acid (LNA) Analogue
gactcagtaagaacacctgtacaacgagaggaa

SEQ ID NO: 139 - DNA, 24 bp, Artificial Sequence
  Lz-B_T547K-WT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctgcctgttaaa

SEQ ID NO: 140 - DNA, 24 bp, Artificial Sequence
  Lz-B_T547K-MT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctgccttttaaa

SEQ ID NO: 141 - DNA, 30 bp, Artificial Sequence
  A570D_WT_analyte_S
cattgctgacactactgatgctgtccgtga

SEQ ID NO: 142 - DNA, 30 bp, Artificial Sequence
  A570D_MT_analyte_S
cattgatgacactactgatgctgtccgtga

SEQ ID NO: 143 - DNA, 90 bp, Artificial Sequence
  A570D_WT_template
tctaacaaaaagtttctgcctttccaacaatttggcagagacattgctgacactactgatgctgtccgtgatccacagacacttgagatt

SEQ ID NO: 144 - DNA, 90 bp, Artificial Sequence
  A570D_MT_template
tctaacaaaaagtttctgcctttccaacaatttggcagagacattgatgacactactgatgctgtccgtgatccacagacacttgagatt

SEQ ID NO: 145 - DNA, 30 bp, Artificial Sequence
  RPA_A570D_RVS
tcaagaatctcaagtgtctgtggatcacgg

SEQ ID NO: 146 - DNA, 55 bp, Artificial Sequence
  RPA_A570D_FWD
  misc_feature (1)..(22): T7 Promoter
gaaattaatacgactcactatagggctgagtctaacaaaaagtttctgcctttcc

SEQ ID NO: 147 - DNA, 35 bp, Artificial Sequence
  Mz-A_A570D-7+8sub
tcacggacagcatcagtagtacaacgagaggaaag

SEQ ID NO: 148 - DNA, 25 bp, Artificial Sequence
  Mz-B_A570D-WT-7+8sub
ccagggaggctagctgtcagcaatg

SEQ ID NO: 149 - DNA, 25 bp, Artificial Sequence
  Mz-B_A570D-MT-7+8sub
ccagggaggctagctgtcatcaatg

SEQ ID NO: 150 - DNA, 33 bp, Artificial Sequence
  Mz-66_A_A570D-FAM
tcacggacagcatcagtagtacaacgaggttag

SEQ ID NO: 151 - DNA, 24 bp, Artificial Sequence
  Mz-66_B_A570D-WT-FAM
tcatgaggctagctgtcagcaatg

SEQ ID NO: 152 - DNA, 24 bp, Artificial Sequence
  Mz-66_B_A570D-MT-FAM
tcatgaggctagctgtcatcaatg

SEQ ID NO: 153 - DNA, 33 bp, Artificial Sequence
  Lz-A_A570D-HEX
  misc_feature (32)..(33): Locked Nucleic Acid (LNA) Analogue
tcacggacagcatcagtagtacaacgagaggaa

SEQ ID NO: 154 - DNA, 24 bp, Artificial Sequence
  Lz-B_A570D-WT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctgtcagcaatg

SEQ ID NO: 155 - DNA, 24 bp, Artificial Sequence
  Lz-B_A570D-MT-HEX
  misc_feature (1)..(2): Locked Nucleic Acid (LNA) Analogue
  misc_feature (19)..(19): Locked Nucleic Acid (LNA) Analogue & SNP site on the analyte
cagggaggctagctgtcatcaatg

SEQ ID NO: 156 - DNA, 33 bp, Artificial Sequence; identical to SEQ ID NO: 150
  Lz-A_A570D-FAM
  misc_feature (32)..(33): Locked Nucleic Acid (LNA) Analogue & SNP site on the analyte

SEQ ID NO: 157 - DNA, 24 bp, Artificial Sequence
  Lz-B_A570D-WT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctgtcagcaatg

SEQ ID NO: 158 - DNA, 24 bp, Artificial Sequence
  Lz-B_A570D-MT-FAM
  misc_feature (1)..(2): Locked Nucleic Acid (LNA) Analogue
  misc_feature (19)..(19): Locked Nucleic Acid (LNA) Analogue & SNP site on the analyte
tcatgaggctagctgtcatcaatg

SEQ ID NO: 159 - DNA, 24 bp, Artificial Sequence
  Lz-B_A570D-WT-FAM_SNP
  misc_feature (19)..(19): Locked Nucleic Acid (LNA) Analogue & SNP site on the analyte
tcatgaggctagctgtcagcaatg

SEQ ID NO: 160 - DNA, 24 bp, Artificial Sequence
  Lz-B_A570D-MT-FAM_SNP
  misc_feature (19)..(19): Locked Nucleic Acid (LNA) Analogue & SNP site on the analyte
tcatgaggctagctgtcatcaatg

SEQ ID NO: 161 - DNA, 30 bp, Artificial Sequence
  P681_WT_analyte
ttctcctcggcgggcacgtagtgtagctag

SEQ ID NO: 162 - DNA, 30 bp, Artificial Sequence
  P681H_MT_analyte
ttctcatcggcgggcacgtagtgtagctag

SEQ ID NO: 163 - DNA, 35 bp, Artificial Sequence
  Mz-A_P681H-7+8sub
ctagctacactacgtgcccgacaacgagaggaaag

SEQ ID NO: 164 - DNA, 25 bp, Artificial Sequence
  Mz-B_P681H-WT-7+8sub
ccagggaggctagctccgaggagaa

SEQ ID NO: 165 - DNA, 25 bp, Artificial Sequence
  Mz-B_P681H-MT-7+8sub
ccagggaggctagctccgatgagaa

SEQ ID NO: 166 - DNA, 30 bp, Artificial Sequence
  D796Y_WT_analyte
ttaaagattttggtggttttaatttttcac

SEQ ID NO: 167 - DNA, 30 bp, Artificial Sequence
  D796Y_MT_analyte
ttaaatattttggtggttttaatttttcac

SEQ ID NO: 168 - DNA, 35 bp, Artificial Sequence
  Mz-A_D796Y-7+8sub
gtgaaaaattaaaaccaccaacaacgagaggaaag

SEQ ID NO: 169 - DNA, 25 bp, Artificial Sequence
  Mz-B_D796Y-WT-7+8sub
ccagggaggctagctaaatctttaa

SEQ ID NO: 170 - DNA, 25 bp, Artificial Sequence
  Mz-B_D796Y-MT-7+8sub
ccagggaggctagctaaatatttaa

SEQ ID NO: 171 - DNA, 30 bp, Artificial Sequence
  D950N_WT_analyte
ttcaagatgtggtcaaccaaaatgcacaag

SEQ ID NO: 172 - DNA, 30 bp, Artificial Sequence
  D950N_MT_analyte
ttcaaaatgtggtcaaccaaaatgcacaag

SEQ ID NO: 173 - DNA, 35 bp, Artificial Sequence
  Mz-A_D950N-7+8sub
cttgtgcattttggttgaccacaacgagaggaaag

SEQ ID NO: 174 - DNA, 25 bp, Artificial Sequence
  Mz-B_D950N-WT-7+8sub
ccagggaggctagctacatcttgaa

SEQ ID NO: 175 - DNA, 25 bp, Artificial Sequence
  Mz-B_D950N-MT-7+8sub
ccagggaggctagctacattttgaa

SEQ ID NO: 176 - DNA, 30 bp, Artificial Sequence
  T1027I_WT_analyte
tgctactaaaatgtcagagtgtgtacttgg

SEQ ID NO: 177 - DNA, 30 bp, Artificial Sequence
  T1027I_MT_analyte
tgctattaaaatgtcagagtgtgtacttgg

SEQ ID NO: 178 - DNA, 35 bp, Artificial Sequence
  Mz-A_T1027I-7+8sub
ccaagtacacactctgacatacaacgagaggaaag

SEQ ID NO: 179 - DNA, 25 bp, Artificial Sequence
  Mz-B_T1027I-WT-7+8sub
ccagggaggctagcttttagtagca

SEQ ID NO: 180 - DNA, 25 bp, Artificial Sequence
  Mz-B_T1027I-MT-7+8sub
ccagggaggctagcttttaatagca

SEQ ID NO: 181 - DNA, 30 bp, Artificial Sequence
  D1118H_WT_analyte
ctacagacaacacatttgtgtctggtaact

SEQ ID NO: 182 - DNA, 30 bp, Artificial Sequence
  D1118H_MT_analyte
ctacacacaacacatttgtgtctggtaact

SEQ ID NO: 183 - DNA, 35 bp, Artificial Sequence
  Mz-A_D1118H-7+8sub
agttaccagacacaaatgtgacaacgagaggaaag

SEQ ID NO: 184 - DNA, 25 bp, Artificial Sequence
  Mz-B_D1118H-WT-7+8sub
ccagggaggctagctttgtctgtag

SEQ ID NO: 185 - DNA, 25 bp, Artificial Sequence
  Mz-B_D1118H-MT-7+8sub
ccagggaggctagctttgtgtgtag

SEQ ID NO: 186 - DNA, 33 bp, Artificial Sequence; identical to SEQ ID NO: 43
  Mz-A
  misc_feature (1)..(20): 2′-fluoroarabino nucleic acid (FANA) residues
  misc_feature (28)..(33): 2′-fluoroarabino nucleic acid (FANA) residues

SEQ ID NO: 187 - DNA, 34 bp, Artificial Sequence
  Mz-B
  misc_feature (1)..(6): 2′-fluoroarabino nucleic acid (FANA) residues
  misc_feature (8)..(8): FANA nucleotide
  misc_feature (14)..(33): 2′-fluoroarabino nucleic acid (FANA) residues
ucaugaggctacgugaagaaucaccaggagucaa

SEQ ID NO: 188 - DNA, 40 bp, Artificial Sequence
  Trigger
uugacuccuggugauucuucuucagguuggacagcuggug

SEQ ID NO: 189 - DNA, 13 bp, Artificial Sequence
  Reporter
cuaaccgucauga